(19) United States
(12) Patent Application Publication
(10) Pub. No.: US 2016/0030518 A1
(43) Pub. Date: Feb. 4, 2016

(54) COMPOSITIONS AND METHODS FOR INHIBITING VIRAL ACTIVITY

(71) Applicant: THE REGENTS OF THE UNIVERSITY OF CALIFORNIA, Oakland, CA (US)

(72) Inventor: Jay A. Levy, San Francisco, CA (US)

(21) Appl. No.: 14/777,388

(22) PCT Filed: Mar. 31, 2014
(86) PCT No.: PCT/US14/32410

§ 371 Date: Sep. 15, 2015

Related U.S. Application Data
(60) Provisional application No. 61/890,630, filed on Oct. 14, 2013.
(60) Provisional application No. 61/808,048, filed on Apr. 3, 2013.

(51) Int. Cl. A61K 38/17; C12N 7/00

(52) U.S. Cl. CPC A61K 38/1793; C12N 7/00; A61K 38/17; C12N 2740/16063; C12N 2710/16163; C12N 2710/16563; C12N 2770/32663; C12N 2770/24163; C12N 2760/18063; C12N 2720/12363; C12N 2770/32763

(57) ABSTRACT

The present disclosure provides methods of inhibiting viral activity in a cell, the methods generally involving contacting the cell with a soluble CD137 polypeptide or a FAM3C polypeptide. The present disclosure provides methods for treating a virus infection in an individual, the methods generally involving administering to the individual an effective amount of a soluble CD137 polypeptide or a FAM3C polypeptide.

### CROSS-REFERENCE

This application claims the benefit of U.S. Provisional Patent Application No. 61/808,048, filed Apr. 3, 2013; and U.S. Provisional Patent Application No. 61/890,630, filed Oct. 14, 2013, which applications are incorporated herein by reference in their entirety.

### STATEMENT REGARDING FEDERALLY SPONSORED RESEARCH

This invention was made with government support under Grant No R01 AI056992 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “UCSF-486WO_SeqList_ST25.txt” created on Mar. 13, 2014 and having a size of 25 KB. The contents of the text file are incorporated by reference herein in their entirety.

### INTRODUCTION

Viral infections, including Human Immunodeficiency Virus (HIV), ebola virus, parvovirus; papillomaviruses, hantaviruses, influenza viruses, hepatitis viruses A to G, etc. have devastating consequences to health. HIV infection in particular is a pressing threat to public health worldwide. According to UNAIDS global estimates, 34.0 million [31.4 million-35.9 million] people were living with HIV at the end of 2011. An estimated 0.8% of adults aged 15-49 years worldwide are living with HIV, although the burden of the epidemic continues to vary considerably between countries and regions.

CD8+ cells from healthy HIV-1 infected individuals can suppress human immunodeficiency virus (HIV) replication in infected CD4+ cells without killing the cell. This CD8+ cell non-cytotoxic antiviral response (CNAR) is not restricted by class I or class II molecules and is mediated at least in part by production of a soluble factor(s) from CD8+ cells. One of these proteins is the CD8⁺ cell antiviral factor (CAF). (Levy (2003) Trends Immunol. 24:628; Walker et al. (1986) Science 234:1563; and U.S. Pat. Nos. 5,565,549; 5,580,769; and 5,707,814).

There is a need in the art for methods of treating viral infections.

### LITERATURE

U.S. Pat. Nos. 5,580,769; 5,707,814; and 5,565,549; U.S. Patent Application US20130101610; Michel et al. (1998) Eur. J. Immunol. January; 28(1):290-5; Goodwin et al. (1993) Eur. J. Immunol. October; 23(10):2631-41; Levy (2003) Trends Immunol. 24:628; Walker et al. (1986) Science 234:1563.

### SUMMARY

The present disclosure provides methods of inhibiting viral activity in a cell, the methods generally involving contacting the cell with a soluble CD137 polypeptide or a FAM3C polypeptide. The present disclosure provides methods for treating a virus infection in an individual, the methods generally involving administering to the individual an effective amount of a soluble CD137 polypeptide or a FAM3C polypeptide.

The present disclosure provides methods of treating an individual to inhibit the activity of a virus, where the methods include contacting a target cell of the individual with (e.g., administering to the individual) an effective amount of an anti-viral composition comprising at least one anti-viral agent selected from: an sCD137 polypeptide, a nucleic acid comprising a nucleotide sequence that encodes an sCD137 polypeptide, a FAM3C polypeptide, and a nucleic acid comprising a nucleotide sequence that encodes a FAM3C polypeptide. The treated individual can be infected with the virus, can have an increased risk of being infected with the virus, or can be suspected of being infected with the virus. In some embodiments, the individual is a human. Some embodiments further include measuring viral activity in a biological sample obtained from the individual, where the biological sample can be obtained from the individual prior to the step of administering and/or after the step of administering.

The present disclosure further provides methods of inhibiting viral activity in a target cell that is infected with a virus, that has an increased risk of acquiring a virus, or that is suspected of being infected with a virus, where the methods include at least one of: (i) contacting the target cell with an effective amount of an anti-viral composition comprising at least one anti-viral agent selected from: an sCD137 polypeptide, and a FAM3C polypeptide; and (ii) introducing into the cell an anti-viral composition comprising at least one anti-viral agent selected from: a nucleic acid comprising a nucleotide sequence that encodes an sCD137 polypeptide, and a nucleic acid comprising a nucleotide sequence that encodes a FAM3C polypeptide. In the context of HIV, target cells include, e.g., CD4⁺ T cells, microglia, astrocytes, brain macrophages, and the like. Other target cells include, e.g., epithelial cells.

In some embodiments, the virus is HIV (Human Immunodeficiency Virus). In some embodiments, the contacting occurs in vivo while in some embodiments, the contacting occurs ex vivo. In some embodiments, the target cell is contacted with the anti-viral composition up to about 48 hours prior to suspected potential contact with the virus, up to about 4 hours after being contacted with the virus, or up to about 4 hours after suspected contact with the virus. Some embodiments further include measuring viral activity (e.g., prior to the step of contacting, after the step of contacting, or both prior to and after the step of contacting the target cell with an anti-viral composition).

### BRIEF DESCRIPTION OF THE DRAWINGS

FIG. 1 depicts the anti-viral activity of CD8⁺ cells transfected to express sCD137 (soluble CD137) or FAM3C. A CNAR assay was performed.

FIG. 2 depicts data showing that the supernatant (i.e., conditioned media) from sCD137- or FAM3C-transfected CD8⁺ cells showed anti-HIV activity when added to primary CD4⁺ cells acutely infected with HIV.

FIG. 3 depicts the results from experiments used to determine the effective dose (ED50) of sCD137 produced by sCD137-transfected HEK (Human Embryonic Kidney) 293 cells for inhibiting HIV activity in primary CD4+ cells.

FIG. 4 depicts data showing that the fusion protein sCD137-Fc (also called TNFRSF9-Fc) retained the anti-HIV activity of the untagged protein.

FIG. 5 depicts data showing that sCD137 supernatant from transfected HEK293 cells showed anti-HIV activity against a wide variety of HIV viruses, including HIV-1 of clades A, B, and C, as well as HIV-2.

FIG. 6 depicts data showing that monoclonal antibody (mAb) against sCD137 neutralizes the antiviral effect of supernatant from sCD137-transfected cells when assayed on acutely infected CD4+ cells.

FIG. 7 depicts data showing that when the sCD137-Fc is removed (depleted; dep) from a supernatant the antiviral activity of sCD137 is also removed.

FIG. 8 depicts purification of sCD137 using QHP and HIC column chromatography.

FIGS. 9A-B depict (A) evidence that sCD137 was purified and (B) evidence that purified sCD137 (sCD137-Fc) has anti-viral activity.

FIG. 10 depicts sCD137 levels in CD8+ cell supernatants from HIV-infected long-term survivors (LTS) and normal (NL) subjects.

FIG. 11 depicts data showing that depletion of sCD137 with antibody (Ab) from a CAF-containing supernatant does not reduce the antiviral activity of the CAF-containing supernatant.

FIGS. 12A-C depict data showing that the anti-HIV effect is pronounced when cells are treated for up to 6 hours prior to viral infection or up to 4 hours after viral infection. Pre-treatment with sCD137 established an antiviral state that lasted for at least 48 hours.

FIG. 13 depicts data showing that high level of membrane-bound CD137 is seen on CD8+ cells and lower levels on CD4⁺ T cells.

FIG. 14 depicts data showing that very low levels of the CD137 ligand (CD137L) were detected on CD4⁺ T cells.

FIG. 15 depicts data showing that that primary CD4⁺ T cells can absorb or remove sCD137 from the sCD137-containing supernatants.

FIG. 16 depicts data showing that a secretion by CD4+cells is not needed for the antiviral activity of sCD137.

FIG. 17 depicts data showing that a soluble form of CD137 ligand (CD137L) does not block the activity of sCD137 (see also, example 5-Table 2).

FIG. 18 depicts data showing that FAM3C and FAM3C linked to a V5 tag (FAM3C-v5) suppresses CD4+ cells acutely infected with HIV.

FIG. 19 depicts data showing that mixing sCD137 and FAM3C did not change the anti-HIV activity of either one of the proteins.

FIG. 20 depicts data showing that when human U373 glioblastoma cells (MAGI), expressing either the R5 or X4 chemokine receptor, were inoculated with HIV (either R5- or X4-tropic) and then supernatant was added, virus replication was inhibited by sCD137-containing supernatant, but not by FAM3C-containing supernatant.

FIGS. 21A-B depict data showing the effect of sCD137 on transcription in CD4⁺T and HeLa cells, and showing that the block of viral activity by sCD137 resembles that induced by interferon-α.

FIG. 22 depicts data showing that sCD137 inhibits poliovirus activity in HeLa cells infected with poliovirus.

FIGS. 23A-D depict inhibition of polio virus activity in Vero cells (African green monkey kidney cells). (FIG. 23C is HIV).

FIG. 24 depicts data showing that sCD137 inhibits the activity of multiple different flaviviruses in Vero cells (African green monkey kidney cells).

FIGS. 25A-B depict data showing that sCD137 inhibits NiV virus activity, LASV virus activity, and ZEBOV virus activity in Human Umbilical Vein Endothelial Cells (HUVECs); and LASV activity in Vero cells and Hep G2 cells.

FIG. 26 depicts data showing that sCD137 is stable at different temperatures.

FIGS. 27A-B provide an alignments of the human and mouse CD137 (TNFRSF9) proteins. (A) “Query 1” (SEQ ID NO: 24) is amino acids 1 to 205 of mouse CD137 (SEQ ID NO: 22). “Sbjct 1” (SEQ ID NO: 25) is amino acids 1 to 252 of human CD137 (SEQ ID NO:1). (B) “Query 1” (SEQ ID NO: 27) is amino acids 1 to 138 of mouse CD137 (SEQ ID NO: 22). “Sbjct 1” (SEQ ID NO: 23) is amino acids 1 to 138 of human CD137 (SEQ ID NO:1).

### DEFINITIONS

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

A “biological sample” encompasses a variety of sample types obtained from an individual. The definition encompasses blood, serum, plasma, and other liquid samples of biological origin (e.g., seminal fluid, i.e., semen, vaginal fluid, saliva, urine, sweat, and the like); solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as epithelial cells. The term “biological sample” encompasses a clinical sample, and also includes cells in culture, cell supernatants, organs, tissue samples, biopsy samples (e.g., lung biopsy samples), epithelial cells (e.g., lung epithelial cells, gastrointestinal epithelial cells, etc.), gastrointestinal tract tissue samples, bronchoalveolar lavage (BAL) fluid samples, nasal lavage fluid samples, blood, plasma, serum, cerebrospinal fluid, seminal fluid, vaginal fluid, saliva, urine, sweat, fecal samples, and the like.

As used herein, the terms “treatment,” “treating,” and the like, refer to obtaining a desired pharmacologic and/or physiologic effect. The effect may be prophylactic in terms of completely or partially preventing a disease (e.g., viral infection) or symptom thereof and/or may be therapeutic in terms of a partial or complete cure for a disease (e.g., viral infection) and/or adverse effect attributable to the disease (e.g., viral infection). “Treatment,” as used herein, covers any treatment of a disease in a mammal, particularly in a human, and includes: (a) preventing the disease (e.g., viral infection) from occurring in a subject which may be predisposed to the disease (e.g., viral infection) but has not yet been diagnosed as having it; (b) inhibiting the disease (e.g., viral infection), i.e., arresting its development; and (c) relieving the disease (e.g., viral infection), i.e., causing regression of the disease (e.g., viral infection).

A “target cell” is a cell that is infected with a virus, is suspected of being infected with a virus, is a cell with increased susceptibility for being infected by a virus, or is a cell at risk for being infected with a virus (e.g., due to current or future exposure to a virus).

The terms “individual,” “subject,” “host,” and “patient,” used interchangeably herein, refer to a mammal, including, but not limited to rodents (e.g., rats, mice), non-human primates, humans, canines, felines, ungulates (e.g., equines, bovines, ovines, porcines, caprines), etc. In some embodiments, the subject has a viral infection (i.e., at least one of the subject's cells is infected with a virus). In some embodiments, the subject does not have a viral infection, but has an increased risk of being infected with the virus (e.g., is at risk for exposure to a viral infection, or is at risk of contracting a viral infection due to increased susceptibility). In some embodiments, it is unknown whether the subject has a viral infection, and in some embodiments, the subject is suspected of having a viral infection or suspected of having an increased risk of contracting a viral infection. An individual with an increased risk of being infected with the virus or an increased risk of contracting a viral infection has an “increased risk” relative to the general population.

A “therapeutically effective amount” or “efficacious amount” refers to the amount of a compound that, when administered to a mammal or other subject for treating a disease (e.g., viral infection), is sufficient to effect such treatment for the disease (e.g., viral infection). The “therapeutically effective amount” will vary depending on the compound or the cell, the disease (e.g., viral infection) and its severity and the age, weight, etc., of the subject to be treated.

The terms “co-administration” and “in combination with” include the administration of two or more therapeutic agents either simultaneously, concurrently or sequentially within no specific time limits In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

The terms “inhibiting”, “reducing”, “suppressing”, and the like in the context of inhibiting viral activity, are used interchangeably to mean causing or leading to a reduction in viral activity.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a sCD137 protein” includes a plurality of such sCD137 proteins and reference to “the sCD137 protein” includes reference to one or more sCD137 proteins and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Provided herein are methods of treating an individual to inhibit the activity of a virus and methods of inhibiting viral activity in a target cell. The methods include contacting a target with an anti-viral composition comprising an anti-viral agent.

### Viruses

The activity of a variety of different viruses can be inhibited by the subject methods. Examples of viruses that can be inhibited by the subject methods include, but are not limited to: DNA viruses; dsDNA viruses (e.g. enveloped viruses, e.g., herpesviruses; non-enveloped viruses, e.g., adenoviruses; poxviruses, etc.); ssDNA viruses; RNA viruses; dsRNA viruses; ssRNA viruses (e.g., (+)ssRNA viruses, (−)ssRNA viruses, ssRNA-RT viruses, retroviruses, orthomyxoviruses, paramyxoviruses, etc.); HIV (Human immunodeficiency virus, including HIV-1 and HIV-2); parvoviruses; papillomaviruses; hantaviruses; arenoviruses (a type of ssRNA virus) (Lassa Fever virus: Lassa virus (LASV)); Rhabdoviridae (e.g., Vesicular stomatitis virus); pox viruses (Poxyviridae) (e.g., Vaccinia virus); paramyxoviruses (a type of ssRNA virus)(e.g., human respiratory syncytial virus (RSV), Aquaparamyxovirus, Avulavirus, Ferlavirus, Henipavirus, Hendravirus, Nipahvirus (NiV), Morbillivirus, Measles virus, Rinderpest virus, Canine distemper virus, phocine distemper virus, Ovine rinderpest virus, Respirovirus, Sendai virus, Human parainfluenza viruses 1 and 3, as well some of the viruses of the common cold, Rubulavirus, Mumps virus, Achimota virus 1 and 2, Human parainfluenza viruses 2 and 4, Simian parainfluenza virus 5, Menangle virus, Tioman virus, Tuhokovirus 1, 2 and 3, TPMV-like viruses, Tupaia paramyxovirus, Mossman virus, Nariva virus, Salem virus, Species Beilong virus, etc.); orthomyxoviruses (a type of ssRNA virus)(e.g., influenza virus types A and/or B and/or C, Isavirus, Thogotovirus, etc.); filoviruses (a type of ssRNA virus)(e.g., Ebola); flaviviruses (a type of ssRNA virus) (e.g., dengue virus (DENV) (e.g., DENV-1, -2, -3, -4, etc.)), yellow fever virus (YFV), Japanese encephalitis virus (JEV), West Nile virus, tick-borne encephalitis virus, and the like); hepatitis viruses A to G; caliciviruses; astroviruses; Reoviridae (e.g., rotaviruses (a dsRNA virus)); coronaviruses (e.g., human respiratory coronavirus, Middle East respiratory syndrome (MERS) virus, and the like); SV40; picornaviruses (a type of ssRNA virus) (e.g., human rhinoviruses (e.g., A-C), hepatitis A virus (HAV), Foot-and-mouth disease virus (FMDV), poliovirus, enterovirus; coxsackievirus, and the like); Nipah virus (NiV); Lassa virus (LASV); ebola virus (e.g., Zaire ebolavirus (ZEBOV)); hendra virus; human adenovirus; Adenoviridae (e.g., Adenovirus 5); human respiratory syncytial virus (RSV); herpesviruses (e.g., Epstein-Barr virus (EBV), varicella zoster virus, cytomegalovirus (CMV), herpes simplex virus (HSV) (human herpes virus, HHV) (e.g., HSV-1 and-2, HHV-1, -2, -3, -4, -5, -6, -7, -8, etc.)); etc. In some embodiments, the virus of the subject methods is selected from the group consisting of: HIV, DENV (e.g., -1, -2, -3, -4), YFV, JEV, poliovirus, rhinovirus, coxsackievirus, NiV, ZEBOV, CMV, HHV (e.g., -1, -2, -3, -4, -5, -6, -7, -8), and LASV. In some embodiments, the virus is HIV.

### Target Cells

A “target cell” is a cell that is infected with a virus, is a cell that is suspected of being infected with a virus, is a cell that is infected with a virus, is a cell that has an increased risk of acquiring a virus (e.g., has an increased susceptibility for being infected by a virus; is a cell at risk for being infected with a virus (e.g., due to increased chance of current or future exposure to a virus); etc.), or is a cell suspected of having a virus (e.g., a cell suspected of being infected with a virus). Thus, in some embodiments, the subject methods are used to prevent infection (e.g., of a target cells, of an individual, etc.). In some embodiments (e.g., in vivo embodiments) a target cell is in close association with non-target cells.

A target cell can be any cell type that can be infected by a given virus. For example, in embodiments where the subject virus is HIV, the target cell can be a CD4⁺ T cell because HIV infects CD4⁺ T cells; in embodiments where the subject virus is RSV, the target cell can be respiratory epithelial cell; in embodiments where the subject virus is poliovirus, the target cell can be a CD155⁺ cell and/or a cell from a cell line (e.g., a HeLa cell); in embodiments where the subject virus is EBV, the target cell can be, for example, a B-lymphocyte or epithelial cell; in embodiments where the subject virus is HSV, the target cell can be a mucoepithelial cell; and in embodiments where the subject virus is CMV, the target cell can be a monocyte, a lymphocyte, an epithelial cell, and/or a cell from a cell line (e.g., human foreskin fibroblasts); in embodiments where the subject virus is influenza virus, the target cell can be a respiratory epithelial cell. Suitable cells include any convenient primate cell. Examples of suitable cells include, but are not limited to: Calu-3 cells (a Homo sapiens lung adenocarcinoma cell line), Human Umbilical Vein Endothelial Cells (HUVECs), Vero cells (African Green Monkey kidney cells), and Hep G2 cells (a human liver epithelial cell line). As demonstrated in the examples below, sCD137 reduces viral activity in a wide variety of cell types (e.g., primary CD4+ T cells, HeLa cells, 293 human embryonic kidney cells (HEK cells), HT-29 human colon cancer cells, Hep2 human liver cells, Human endothelial cells (HUVEC), U373 human glioblastoma cells, and monkey Vero cells).

### Anti-Viral Agents and Compositions

Subject anti-viral agents include sCD137 (soluble CD137), FAM3C, biologically active equivalents, biologically active variants, and biologically active fragments thereof.

The term “CD137” is used herein to refer to the protein CD137 (SEQ ID NO:1) (Uniprot accession number Q07011, NCBI accession number NP_{—}001552.2), which is also known as TNFRSF9 (Tumor Necrosis Factor Receptor Superfamily, Member 9), 4-1BB, CDw137, ILA (Induced By Lymphocyte Activation), 4-1BB ligand receptor; CD137 antigen; T cell antigen ILA; T-cell antigen 4-1BB homolog; T-cell antigen ILA, homolog of mouse 4-1BB, interleukin-activated receptor, homolog of mouse Ly63, and receptor protein 4-1BB. CD137 can be expressed by activated T cells, but to a greater extent by CD8+ T cells than CD4+ T cells.

An example of an amino acid sequence of a naturally occurring human CD137 is:

(SEQ ID NO: 1)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCEL

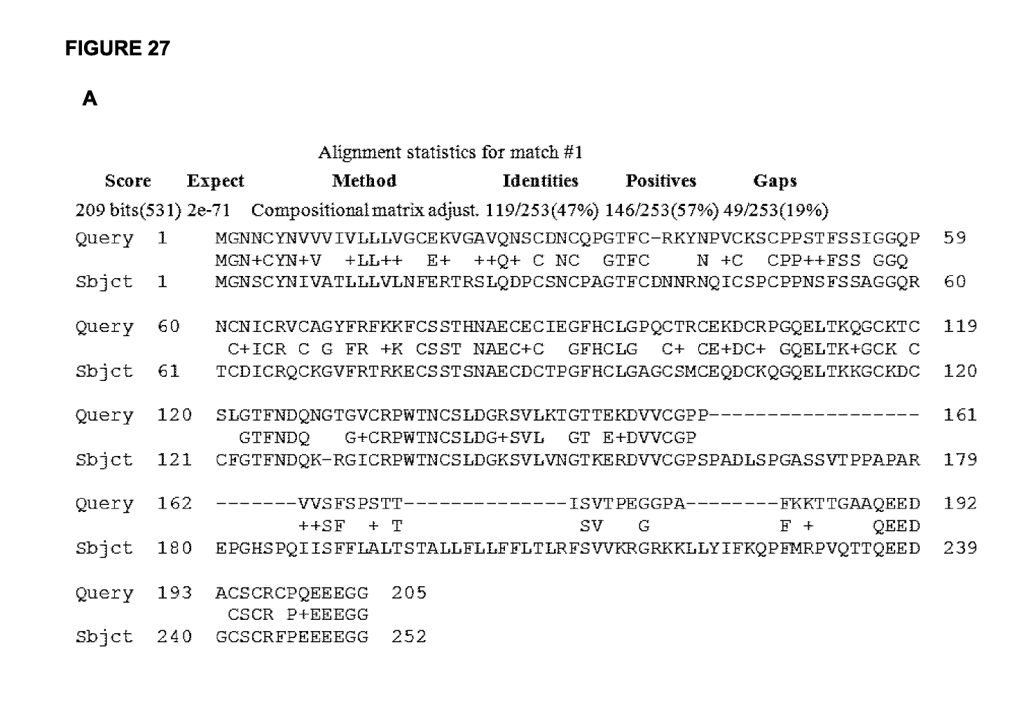

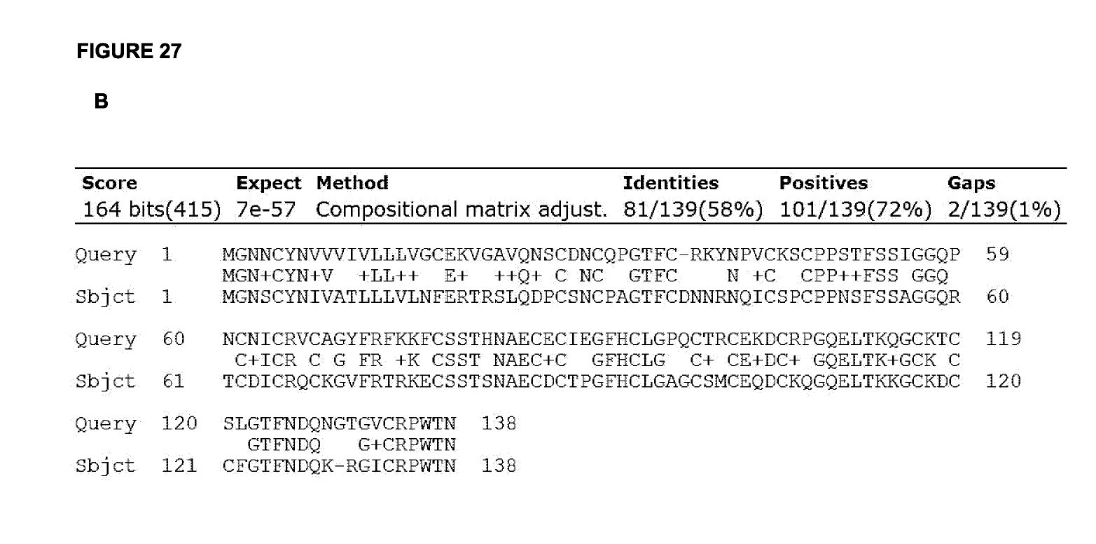

Structural features of CD137 include a signal peptide (amino acids 1-17); a Tumor Necrosis Factor Receptor (TNFR) domain (amino acids 19-106); a TNFR-Cys 1 region (amino acids 24-45); a TNFR-Cys 2 region (amino acids 47-86); a TNFR-Cys 3 region (amino acids 87-118); a TNFR-Cys 4 region (amino acids 119-159); a transmembrane region (amino acids 187-213); and a region identified to interact with LRR-1 (amino acids 214-255) Amino acids N-terminal to the transmembrane domain are extracellular while amino acids C-terminal to the transmembrane domain are intracellular. FIG. 27 provides alignments of the human and mouse CD137 (TNFRSF9) proteins (“Query 1” is mouse and “Sbjct 1” is human).

An example of a nucleotide sequence encoding a naturally occurring human CD137 is:

(SEQ ID NO: 2)
ATGGGAAACAGCTGTTACAACATAGTAGCCACTCTGTTGCTGGTCCTCA
ACTTTGAGAGGACAAGATCATTGCAGGATCCTTGTAGTAACTGCCCAGC
TGGTACATTCTGTGATAATAACAGGAATCAGATTTGCAGTCCCTGTCCT
CCAAATAGTTTCTCCAGCGCAGGTGGACAAAGGACCTGTGACATATGCA
GGCAGTGTAAAGGTGTTTTCAGGACCAGGAAGGAGTGTTCCTCCACCAG
CAATGCAGAGTGTGACTGCACTCCAGGGTTTCACTGCCTGGGGGCAGGA
TGCAGCATGTGTGAACAGGATTGTAAACAAGGTCAAGAACTGACAAAAA
AAGGTTGTAAAGACTGTTGCTTTGGGACATTTAACGATCAGAAACGTGG
CATCTGTCGACCCTGGACAAACTGTTCTTTGGATGGAAAGTCTGTGCTT
GTGAATGGGACGAAGGAGAGGGACGTGGTCTGTGGACCATCTCCAGCCG
ACCTCTCTCCGGGAGCATCCTCTGTGACCCCGCCTGCCCCTGCGAGAGA
GCCAGGACACTCTCCGCAGATCATCTCCTTCTTTCTTGCGCTGACGTCG
ACTGCGTTGCTCTTCCTGCTGTTCTTCCTCACGCTCCGTTTCTCTGTTG
TTAAACGGGGCAGAAAGAAACTCCTGTATATATTCAAACAACCATTTAT
GAGACCAGTACAAACTACTCAAGAGGAAGATGGCTGTAGCTGCCGATTT
CCAGAAGAAGAAGAAGGAGGATGTGAACTG.

The terms “sCD137” and “soluble CD137” are used interchangeably herein to refer to a soluble form of the CD137 protein. Two natural variants of sCD137 have been identified that are isoforms of CD137, where both short forms appear to lead to frame shifts that cause the elimination of the transmembrane domain of the CD137 protein (Michel et al., Eur J Immunol 1998; 28: 290-295, which is herein incorporated by reference in its entirety). The two forms of sCD137 have deletions in nucleotide 414 to nucleotides 545 and 681, respectively. As stated above, the deletions within the natural occurring isoforms of sCD137 lead to frame shifts. Thus, both naturally occurring sCD137 proteins retain the extracellular amino acids 1-138 of CD137 (SEQ ID NO:1), which are N-terminal to the transmembrane domain, and result in the addition of a small number of amino acids (which are encoded by a small number of codons prior to a frame-shift induced stop codon) that are not present in CD137. The terms “sCD137” and “soluble CD137” encompass both naturally occurring variants described in Michel et al, both of which can be suitable anti-viral agents in the subject methods. In addition, the terms “sCD137” and “soluble CD137” also encompass artificial forms of sCD137 comprising the extracellular amino acids 1-138 of CD137 (SEQ ID NO:1). A polypeptide comprising amino acids 1-190 of CD137 (SEQ ID NO:1) can also be a suitable sCD137. For example, one example of a suitable sCD137 is (see examples section for working examples that use the nucleic acid sequence set forth as SEQ ID NO:21 to express the protein set forth as SEQ ID NO:20):

Amino acid sequence:
(SEQ ID NO: 20)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISF
(amino acids 1-190 of human CD137, SEQ ID NO: 1)
Nucleotide sequence:
(SEQ ID NO: 21)
Atgggaaacagctgttacaacatagtagccactctgdgctggtcctca
actagagaggacaagatcattgcaggatccttgtagtaactgcccagc
tggtacattctgtgataataacaggaatcagatttgcagtccctgtcc
tccaaatagtactccagcgcaggtggacaaaggacctgtgacatatgc
aggcagtgtaaaggtgattcaggaccaggaaggagtgacctccaccag
caatgcagagtgtgactgcactccagggtacactgcctgggggcagga
tgcagcatgtgtgaacaggattgtaaacaaggtcaagaactgacaaaa
aaaggttgtaaagactgttgctttgggacatttaacgatcagaaacgt
ggcatctgtcgaccctggacaaactgactaggatggaaagtctgtgct
tgtgaatgggacgaaggagagggacgtggtctgtggaccatctccagc
cgacctctctccgggagcatcctctgtgaccccgcctgcccctgcgag
agagccaggacactctccgcagatcatctccactag

Another exemplary sCD137 is:

(SEQ ID NO: 23)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTN
(amino acids 1-138 of human CD137, SEQ ID NO: 1).

One may refer to the CD137 sequence alignments between the mouse and human CD137 proteins (FIG. 27) to determine exemplary sCD137 proteins that can be derived from the mouse CD137 sequence. For example, a mouse derived exemplary sCD137, corresponding to amino acids 1-190 of human CD137, is:

(SEQ ID NO: 26)
MGNNCYNVVVIVLLLVGCEKVGAVQNSCDNCQPGTFCRKYNPVCKSCPPS
TFSSIGGQPNCNICRVCAGYFRFKKFCSSTHNAECECIEGFHCLGPQCTR
CEKDCRPGQELTKQGCKTCSLGTFNDQNGTGVCRPWTNCSLDGRSVLKTG
TTEKDVVCGPPVVSF
(amino acids 1-165 of mouse CD137, SEQ ID NO: 22).

Another mouse derived exemplary sCD137, corresponding to amino acids 1-138 of human CD137, is:

(SEQ ID NO: 27)
MGNNCYNVVVIVLLLVGCEKVGAVQNSCDNCQPGTFCRKYNPVCKSCPP
STFSSIGGQPNCNICRVCAGYFRFKKFCSSTHNAECECIEGFHCLGPQC
TRCEKDCRPGQELTKQGCKTCSLGTFNDQNGTGVCRPWTN
(amino acids 1-138 of mouse CD137, SEQ ID NO: 22).

The term “sCD137” as used herein also encompasses biologically active variants (including natural homologs) of the naturally occurring sCD137 proteins described above as well as biologically active variants of artificial sCD137 proteins described above. Suitable sCD137 anti-viral agents comprise an amino acid sequence having 60% or more amino acid sequence identity (e.g., 65% or more, 70% or more, 75% or more, 80% or more, 85% or more, 90% or more, 95% or more, 99% or more, 99.5% or more, or 100%) to amino acids 1-138 of an amino acid sequence set forth in any of SEQ ID NOs: 1, 20, and 22-27. A sCD137 polypeptide can comprise the amino acid sequence of a naturally-occurring CD137 variant.

The term “FAM3C” is used herein to refer to the secreted protein FAM3C (Family With Sequence Similarity 3, Member C) (SEQ ID NO:3) (Uniprot accession number Q92520, NCBI accession number NP_{—}055703.1 and NP001035109.1), which is also known as GS3786; ILEI (Interleukin-like EMT Inducer); and Predicted Osteoblast Protein.

An example of an amino acid sequence of a human FAM3C is:

(SEQ ID NO: 3)
MRVAGAAKLVVAVAVFLLTFYVISQVFEIKMDASLGNLFARSALDTAARS
TKPPRYKCGISKACPEKHFAFKMASGAANVVGPKICLEDNVLMSGVKNNV
GRGINVALANGKTGEVLDTKYFDMWGGDVAPFIEFLKAIQDGTIVLMGTY
DDGATKLNDEARRLIADLGSTSITNLGFRDNWVFCGGKGIKTKSPFEQHI
KNNKDTNKYEGWPEVVEMEGCIPQKQD

An example of an amino acid sequence of a mouse FAM3C is:

(SEQ ID NO: 28)
MRVAGAAKLVVAVAVFLLTFYVISQVFEIKMDASLGNLFARSALDSAIR
STKPPRYKCGISKACPEKHFAFKMASGAANVVGPKICLEDNVLMSGVKN
NVGRGINIALVNGKTGEVIDTKFFDMWGGDVAPFIEFLKTIQDGTVVLM
ATYDDGATKLTDEARRLIAELGSTSITSLGFRDNWVFCGGKGIKTKSPF
EQHIKNNKETNKYEGWPEVVEMEGCIPQKQD

The term FAM3C as used herein also encompasses biologically active variants (including natural homologs) of the naturally occurring human FAM3C protein (SEQ ID NO:3). Suitable FAM3C anti-viral agents comprise an amino acid sequence having 75% or more, 80% or more, 85% or more, 90% or more, 95% or more, 99% or more or 100% amino acid sequence identity to a contiguous stretch of from about 50 amino acids (aa) to about 100 aa, from about 100 aa to about 150 aa, from about 150 aa to about 200 aa, or from about 200 aa to about 227 aa, of the FAM3C amino acid sequence set forth in SEQ ID NO:3. In some cases, the FAM3C polypeptide comprises the amino acid sequence of a naturally-occurring variant FAM3C.

An example of a nucleotide sequence encoding a human FAM3C is:

(SEQ ID NO: 4)
ATGAGGGTAGCAGGTGCTGCAAAGTTGGTGGTAGCTGTGGCAGTGTTT
TTACTGACATTTTATGTTATTTCTCAAGTATTTGAAATAAAAATGGAT
GCAAGTTTAGGAAATCTATTTGCAAGATCAGCATTGGACACAGCTGCA
CGTTCTACAAAGCCTCCCAGATATAAGTGTGGGATCTCAAAAGCTTGC
CCTGAGAAGCATTTTGCTTTTAAAATGGCAAGTGGAGCAGCCAACGTG
GTGGGACCCAAAATCTGCCTGGAAGATAATGTTTTAATGAGTGGTGTT
AAGAATAATGTTGGAAGAGGGATCAATGTTGCCTTGGCAAATGGAAAA
ACAGGAGAAGTATTAGACACTAAATATTTTGACATGTGGGGAGGAGAT
GTGGCACCATTTATTGAGTTTCTGAAGGCCATACAAGATGGAACAATA
GTTTTAATGGGAACATACGATGATGGAGCAACCAAACTCAATGATGAG
GCACGGCGGCTCATTGCTGATTTGGGGAGCACATCTATTACTAATCTT
GGTTTTAGAGACAACTGGGTCTTCTGTGGTGGGAAGGGCATTAAGACA
AAAAGCCCTTTTGAACAGCACATAAAGAACAATAAGGATACAAACAAA
TATGAAGGATGGCCTGAAGTTGTAGAAATGGAAGGATGCATCCCCCAG
AAGCAAGAC.

In some embodiments, a subject anti-viral agent is a fusion protein, e.g., a polypeptide that comprises sCD137 or FAM3C and a heterologous polypeptide, where the heterologous polypeptide can be referred to as a “fusion partner.” In some such cases, the fusion partner sequence can provide a tag for ease of tracking or purification (e.g., a fluorescent protein, e.g., green fluorescent protein (GFP), a yellow fluorescent protein (YFP), a red fluorescent protein (RFP), a blue fluorescent protein (CFP), mCherry, tdTomato, and the like; a HIS tag, e.g., a 6× His tag; a hemagglutinin (HA) tag; a FLAG tag; a Myc tag; Fc tag, v5 tag, and the like). Suitable epitope tags include, e.g., GST, hemagglutinin (HA; e.g., YPYDVPDYA; SEQ ID NO:17), FLAG (e.g., DYKDDDDK; SEQ ID NO:18), c-myc (e.g., EQKLISEEDL; SEQ ID NO:19), and the like.

A subject anti-viral agent may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, an anti-viral agent can have a covalently linked poly(ethylene glycol) (PEG) group, where the PEG moiety provides for enhanced lifetime in the blood stream.

Additionally or alternatively, a subject anti-viral agent may be fused to a polypeptide permeant domain to promote uptake by the cell. A number of permeant domains are known in the art and may be used, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO:5). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

A subject anti-viral agent may be produced in vitro or by eukaryotic cells or by prokaryotic cells, and it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the present disclosure are anti-viral agents that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The anti-viral agents may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The anti-viral agents can be isolated and purified in accordance with convenient methods known to one of ordinary skill in the art. For example, supernatant (conditioned media) may be prepared from an expression host (e.g., CD8+ lymphocyte, HEK293 cells, etc.) that expresses the anti-viral agent. As non-limiting examples, an expression host (i.e., host cell) can express the anti-viral agent naturally (i.e., without intervention), the expression host can be stimulated to express the anti-viral agent (e.g., with cytokines, small molecules, growth factors, etc.), or the expression host can be genetically modified (e.g., with a nucleic acid or nucleic acids comprising a nucleotide sequence encoding a sCD137 and/or a FAM3C polypeptide) to express a subject anti-viral agent.

A cell has been “genetically modified” or “transformed” or “transfected” by an exogenous nucleic acid (e.g. a recombinant expression vector, isolated mRNA, etc.) when such nucleic has been introduced inside the cell. The presence of exogenous nucleic acid (e.g., nucleic acid or nucleic acids comprising a nucleotide sequence encoding a sCD137 and/or a FAM3C polypeptide) results in permanent or transient genetic change. The transforming nucleic acid may or may not be integrated (covalently linked) into the genome of the cell (e.g., the transforming nucleic acid may be maintained on an episomal elelment such as a plasmid). A stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA.

In the subject methods, the expression host can be any convenient eukaryotic cell type (e.g., a cell of a cell line, e.g., a HeLa cell, a CD8+ cell; or a primary cell, e.g., a CD4+ lymphocyte, a CD8+ lymphocyte, etc.). In some embodiments, an expression host (e.g., a CD8+ lymphocyte) is isolated from a subject individual and is transfected with a nucleic acid comprising a nucleotide sequence encoding a sCD137. In some embodiments, an expression host (e.g., a CD8+ lymphocyte) is isolated from a subject individual and is transfected with a nucleic acid comprising a nucleotide sequence encoding a FAM3C polypeptide. In some embodiments, a genetically modified expression host (expressing an anti-viral agent) is introduced back into the individual from which the cell (e.g., a CD8+ cell) was isolated.

Suitable methods of genetic modification (also referred to as “transformation”) include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995. In cases where an expression host is a CD8+ lymphocyte, suitable methods for transfection include those described in Liu, et., al., J Immunol Methods. 2011 Sep. 30; 372(1-2):22-9, the contents of which are hereby incorporated by reference in their entirety.

A subject anti-viral agent can be purified using HPLC, exclusion chromatography, QHP (ion exchange QHP chromatography), HIC (Hydrophobic Interaction Chromatography) column chromatography, gel electrophoresis, affinity chromatography, or other convenient purification techniques known to one of ordinary skill in the art. In some cases, a polynucleotide encoding an anti-viral agent is introduced into a host cell (i.e., expression host) and the anti-viral agent is purified from the supernatant (conditioned media) using QHP chromatography and HIC chromatography. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Polynucleotides

The disclosure also provides polynucleotides comprising a nucleotide sequence encoding an anti-viral agent (e.g., an sCD137 polypeptide and/or a FAM3C polypeptide) described herein. In some cases, for example, where a polynucleotide comprising a nucleotide sequence encoding an sCD137 polypeptide and/or a FAM3C polypeptide is administered to an individual or is contacted with a target cell (e.g., introduced into a target cell), the polynucleotide can be considered to be a subject an anti-viral agent. In some cases, a polynucleotide comprising a nucleotide sequence encoding an sCD137 polypeptide and/or a FAM3C polypeptide is an anti-viral agent and is administered to an individual. In some cases, a polynucleotide comprising a nucleotide sequence encoding an sCD137 polypeptide and/or a FAM3C polypeptide is an anti-viral agent and is contacted with a target cell (e.g., a CD8+ lymphocyte) isolated from an individual. Suitable methods for transfection of CD8+ lymphocytes include those described in Liu, et., al., J Immunol Methods. 2011 Sep. 30; 372(1-2):22-9, the contents of which are hereby incorporated by reference in their entirety.

In some embodiments, the polynucleotide comprises an expression cassette. In some embodiments, the polynucleotide is a vector comprising the above-described nucleic acid. In some embodiments, the nucleic acid encoding a sCD137 and/or FAM3C polypeptide of the disclosure is operably linked to a transcriptional control element (e.g., a promoter). Promoters are well known in the art. Transcriptional control elements, or promoters, which are useful to drive expression of a subject anti-viral agent or variant thereof in a specific animal cell are numerous and familiar to those skilled in the art. Any promoter that functions in the host cell (i.e., expression host) can be used for expression of an anti-viral agent and/or any variant thereof of the present disclosure. In one embodiment, the promoter used to drive expression of an anti-viral agent can be a promoter that is specific to the expression host cell type. For example, if the expression host is a CD8+ cell (e.g., a CD8+ lymphocyte), a CD8-cell specific promoter (e.g., the promoter of CD8) is a possible suitable promoter for driving expression of a subject anti-viral agent. In some embodiments, the promoter used to drive expression of anti-viral agent can be the EFl a promoter, a cytomegalovirus (CMV) promoter, the CAG promoter, or any other promoter capable of driving expression of a subject anti-viral agent in the expression host.

Also provided herein are vectors comprising a nucleotide sequence encoding an anti-viral agent or any variant thereof described herein. The vectors that can be administered according to the present invention also include vectors comprising a nucleotide sequence which encodes an RNA (e.g., an mRNA) that when transcribed from the polynucleotides of the vector will result in the accumulation of an anti-viral agent in the medium contacting the expression host cells. Vectors which may be used, include, without limitation, lentiviral, Herpes simplex virus (HSV), Cytomegalovirus (CMV), adenoviral, and adeno-associated viral (AAV) vectors. Lentiviruses include, but are not limited to HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of each of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Methods of preparing AAV vectors in a baculovirus system are described in, e.g., WO 2008/024998. AAV vectors can be self-complementary or single-stranded. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos.: 91/18088 and WO 93/09239; U.S. Pat. Nos. 4,797,368, 6,596,535, and 5,139,941; and European Patent No.: 0488528, all of which are hereby incorporated by reference herein in their entireties). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the present disclosure can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the present disclosure are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the present disclosure includes a recombinant virus particle (recombinant because it contains a recombinant polynucleotide) comprising any of the vectors described herein. Methods of producing such particles are known in the art and are described in U.S. Pat. No. 6,596,535, the disclosure of which is hereby incorporated by reference in its entirety.

### Conjugates/Fusions

Another possible modification of a subject nucleic acid and/or subject polypeptide involves chemically linking to the polynucleotide and/or polypeptide one or more moieties or conjugates which enhance the activity, cellular distribution or cellular uptake of the polynucleotide and/or polypeptide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.\

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the N-terminus or C-terminus of a polypeptide (e.g., an sCD137, a FAM3C, etc.). In some embodiments, a PTD is covalently linked to a nucleic acid (e.g., a polynucleotide encoding a CD137, an sCD137, an sFAM3C, a FAM3C, etc.). Exemplary PTDs include but are not limited to a minimal undecapeptide protein transduction domain (corresponding to residues 47-57 of HIV-1 TAT comprising YGRKKRRQRRR; SEQ ID NO:6); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:7); Transportan GWTLNSAGYLLGKINLKALAALAKKIL (SEQ ID NO:8); KALAWEAKLAKALAKALAKHLAKALAKALKCEA (SEQ ID NO:9); and RQIKIWFQNRRMKWKK (SEQ ID NO:10). Exemplary PTDs include but are not limited to, YGRKKRRQRRR (SEQ ID NO:6), RKKRRQRRR (SEQ ID NO:11); an arginine homopolymer of from 3 arginine residues to 50 arginine residues; Exemplary PTD domain amino acid sequences include, but are not limited to, any of the following: YGRKKRRQRRR (SEQ ID NO:6); RKKRRQRR (SEQ ID NO:12); YARAAARQARA (SEQ ID NO:13); THRLPRRRRRR (SEQ ID NO:14); and GGRRARRRRRR (SEQ ID NO:15). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Methods of Inhibiting Viral Activity

The present disclosure provides methods of inhibiting (i.e., suppressing) viral activity (e g , inhibiting viral activity in cells infected with a virus, e.g., human immunodeficiency virus (HIV), or other virus). The methods involve contacting the cell with an anti-viral composition comprising an anti-viral agent, where the anti-viral agent is sCD137, FAM3C, or a combination thereof. Viral activity includes transcription and replication. In some embodiments, a subject method provides for inhibition of viral transcription. In some embodiments, a subject method provides for inhibition of viral replication.

In some cases, a method of the present disclosure can suppress (i.e. inhibit) viral activity in a cell by at least about 10% (10% suppression), at least about 15% (15% suppression), at least about 20% (20% suppression), at least about 25% (25% suppression), at least about 30% (30% suppression), at least about 35% (35% suppression), at least about 40% (40% suppression), at least about 45% (45% suppression), at least about 50% (50% suppression), at least about 60% (60% suppression), at least about 70% (70% suppression), at least about 80% (80% suppression), at least about 90% (90% suppression), at least about 95% (95% suppression), at least about 98% (98% suppression), or more than 98%, compared with the level of viral activity in the cell in the absence of the anti-viral agent.

In some embodiments, an anti-viral agent (e.g., sCD137, FAM3C, or a combination thereof) is co-administered with another anti-viral agent known by one of ordinary skill in the art as appropriate (e.g., co-administer a subject anti-viral agent (sCD137 and/or FAM3C) with an antibody against Ebola virus when ebola virus is the subject virus; co-administer a subject anti-viral agent (sCD137 and/or FAM3C) with an antibody against hendra virus when hendra virus is the subject virus; etc.).

### Methods of Treating an Individual or Inhibiting Viral Activity in a Target Cell

The present disclosure provides methods of treating an individual to inhibit the activity of a virus, the methods generally comprising contacting a target cell of the individual with an effective amount of an anti-viral composition comprising an anti-viral agent that comprises a sCD137 polypeptide, a FAM3C polypeptide, a nucleic acid comprising a nucleotide sequence encoding a sCD137 polypeptide, a nucleic acid comprising a nucleotide sequence encoding a FAM3C polypeptide, or a combination thereof. The present disclosure provides methods of inhibiting the activity of a virus in an individual, the methods generally comprising administering to the individual an effective amount of an anti-viral composition comprising an anti-viral agent that comprises a sCD137 polypeptide, a FAM3C polypeptide, a nucleic acid comprising a nucleotide sequence encoding a sCD137 polypeptide, a nucleic acid comprising a nucleotide sequence encoding a FAM3C polypeptide, or a combination thereof.

An effective amount of anti-viral agent is an amount that inhibits viral activity in target cells by at least about 20%, at least about 30%, at least about 40%, at least about 50%, at least about 60%, at least about 70%, at least about 80%, or at least about 90%, compared to the level of viral activity in the absence of treatment with anti-viral agent. For example, in some cases (e.g., when the individual is suspected of future contact with a virus), the effective amount can be preventative in that it suppresses the amount of viral activity that is present after viral contact (when compared to the amount of viral activity that would otherwise be present in the individual had the individual not been contracted with the anti-viral agent).

In some cases, an effective amount of anti-viral agent is an amount that inhibits viral transcription in an individual by at least about 20% (20% suppression), at least about 30% (30% suppression), at least about 40% (40% suppression), at least about 50% (50% suppression), at least about 60% (60% suppression), at least about 70% (70% suppression), at least about 80% (80% suppression), or at least about 90% (90% suppression), compared to the level of viral transcription in the absence of treatment with anti-viral agent. For example, in some cases (e.g., when the individual is suspected of future contact with a virus), the effective amount can be preventative in that it suppresses the amount of viral transcription that is present after viral contact (when compared to the amount of viral transcription that would otherwise be present in the individual had the individual not been contracted with the anti-viral agent).

In some cases, a subject method involves administering to an individual in need thereof an effective amount of anti-viral agent (e.g., sCD137, FAM3C, or combinations thereof). In some embodiments, an “effective amount” of anti-viral agent (e.g., sCD137, FAM3C, or combinations thereof) is an amount that, when administered to an individual in one or more doses, in monotherapy or in combination therapy, is effective to reduce viral load in the individual by at least about 20% (20% suppression), at least about 30% (30% suppression), at least about 40% (40% suppression), at least about 50% (50% suppression), at least about 60% (60% suppression), at least about 70% (70% suppression), at least about 80% (80% suppression), or at least about 90% (90% suppression), compared to the viral load in the individual in the absence of treatment with anti-viral agent (e.g., sCD137, FAM3C, or combinations thereof). For example, in some cases (e.g., when the individual is suspected of future contact with a virus), the effective amount can be preventative in that it suppresses the viral load that is present after viral contact (when compared to the viral load that would otherwise be present in the individual had the individual not been contracted with the anti-viral agent).

In some embodiments, an “effective amount” of a sCD137 polypeptide or a FAM3C polypeptide is an amount that, when administered in one or more doses to an individual having a virus infection (or to an individual who is suspected of future exposure to a virus), is effective to reduce the number of genome copies of the virus in the individual and/or prevent the number of genome copies of the virus from increasing. In some cases, an effective amount is an amount that will limit or reduce the genome copies/mL serum to a range of from 1000 genome copies/mL serum to 5000 genome copies/mL serum, from 500 genome copies/mL serum to 1000 genome copies/mL serum, from 100 genome copies/mL serum to 500 genome copies/mL serum, or less than 100 genome copies/mL serum. In some embodiments, an “effective amount” of a sCD137 polypeptide or a FAM3C polypeptide is an amount that, when administered in one or more doses to an individual having a virus infection, is effective to achieve a 1.5-log, a 2-log, a 2.5-log, a 3-log, a 3.5-log, a 4-log, a 4.5-log, or a 5-log reduction in viral titer in the serum of the individual.

In some cases, a subject method involves administering to an individual in need thereof (e.g., an individual having a viral infection, an individual expected of being at risk for acquiring a viral infection, etc.) an effective amount of anti-viral agent (e.g., sCD137, FAM3C, or combinations thereof). In some embodiments, an “effective amount” of anti-viral agent (e.g., sCD137, FAM3C, or combinations thereof) is an amount that, when administered to an individual in one or more doses, in monotherapy or in combination therapy, is effective to increase the number of CD4⁺ T cells in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the number of CD4⁺ T cells in the individual in the absence of treatment anti-viral agent (e.g., sCD137, FAM3C, or combinations thereof). For example, in some cases (e.g., when the individual is suspected of future contact with a virus), the effective amount can be preventative in that it suppresses the reduction of CD4+T cells that is present after viral contact (when compared to the reduction of CD4+T cells that would otherwise be present in the individual had the individual not been contracted with the anti-viral agent).

In some embodiments, an effective amount of an antiviral agent (e.g., a sCD137 polypeptide or a FAM3C polypeptide) is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of an antiviral agent (e.g., a sCD137 polypeptide or a

FAM3C polypeptide) is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

For example, Table 1 summarizes results showing that sCD137 inhibits the activity of a wide variety of viruses in multiple different cell types. Table 1 also presents various exemplary ED₅₀ values that correspond to the experiments performed.

TABLE 1
sCD137 has broad and potent antiviral activity.

| | | | | |
|---|---|---|---|---|
| Virus Family | Virus | MOI | Cell Used | ED₅₀ |

Nonsegmented single-stranded RNA viruses

| | | | | |
|---|---|---|---|---|
| Positive sense: | | | | |
| Picornaviridae | Poliovirus | 0.1 | HeLa | 150 ng/ml |
| Flaviviridae | Hepatitis C | 0.1 | Huh-7.5 | 30-50 ng/ml |
| | Dengue | 75** | Vero | 30-70 ng/ml |
| | Japanese | 35** | Vero | <600 |
| | Encephalitis | | | ng/ml*** |
| | Yellow Fever | 35** | Vero | <600 |
| | | | | ng/ml*** |
| Coronaviridae | MERS | 0.1 | Calu-3 | 50-500 ng/ml |
| Retroviridae | HIV | 0.5 | CD4+ T | 250 ng/ml |
| | | | cell | |
| Negative sense: | | | | |
| Rhabdoviridae | Vesicular | 0.01 | Vero | 15-30 ng/ml |
| | stomatitis | | | |
| Paramyxoviridae | Nipah | 0.1 | HepG2 | 300 ng/ml |
| Filoviridae | Ebola | 0.1 | HepG2 | 750 ng/ml |

Segmented single-stranded RNA virus

| | | | | |
|---|---|---|---|---|
| Negative sense: | | | | |
| Orthomyxoviridae | Influenza | | | ND |
| Ambisense: | | | | |
| Arenoviridae | Lassa | 0.01 | HepG2 | 50 ng/ml |

Segmented double-stranded RNA virus

| | | | | |
|---|---|---|---|---|
| Reoviridae | Rotavirus | 0.1 | HT-29 | 100 ng/ml |

Nonsegmented double-stranded DNA viruses

| | | | | |
|---|---|---|---|---|
| Adenoviridae | Adenovirus 5 | 10** | HeLa | 200 ng |
| Herpesviridae | Human | 0.5 | CD4+ T | <2.5 μg/ml*** |
| | Herpesvirus 6 | | cell | |
| | Human | 0.1 | Huf | <2.5 μg/ml*** |
| | Cytomegalovirus | | | |
| Poxviridae | Vaccinia | 1 | Vero | 900 ng/ml |

MOI—multiplicity of infection;
ED₅₀—effective dose that inhibits 50% of infection;
MERS—Middle East respiratory syndrome;
HIV—human immunodeficiency virus;
ND—not done;
*—includes all 4 dengue serotypes;
**focus forming units;
***lowest concentration tested.

In some embodiments, a single dose of an active agent (e.g., a sCD137 polypeptide or a FAM3C polypeptide) is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Administration of an effective amount of a sCD137 polypeptide or a FAM3C polypeptide to an individual in need thereof can result in one or more of: 1) a reduction in viral load; 2) a reduction in viral load in a target biological sample; 3) a reduction in the spread of a virus from one cell to another cell in an individual; 4) a reduction in viral entry into (e.g., reduction of internalization of a virus into) a cell; 5) a reduction in time to seroconversion (virus undetectable in patient serum); 6) an increase in the rate of sustained viral response to therapy; 7) a reduction of morbidity or mortality in clinical outcomes; and 8) an improvement in an indicator of disease response (e.g., a reduction in one or more symptoms of a viral infection, such as fever, etc.).

Any of a variety of methods can be used to determine whether a treatment method is effective. For example, a biological sample obtained from an individual who has been treated with a subject anti-viral composition can be assayed (e.g., where the biological sample was obtained prior to and/or after the initiation of treatment, e.g., prior to and/or after contacting a target cell with a subject anti-viral composition). For example, a biological sample obtained from an individual can be assayed for the presence and/or level of a virus-encoded protein, for the presence and/or level of viral genomes, and the like.

For example, suitable methods of determining whether the methods of the present disclosure are effective in reducing viral load (e.g., HIV viral load), and/or treating a viral infection (e.g., infection with HIV, poliovirus, DENV (e.g., DENV-1, DENV-2, DENV-3, DENV-4, DENV-5), LASV, YSF, ZEBOV, JEV, rhinovirus, coxsackievirus, NiV, CMV, HHV, and the like), include any known test for indicia of virus infection. Suitable tests that measure viral load (i.e, viral titer, viral burden, the amount of virus) in a biological sample include but are not limited to: (i) those that use polymerase chain reaction (PCR) with primers specific for a virus polynucleotide sequence (e.g., HIV, poliovirus, DENV (e.g., DENV-1, DENV-2, DENV-3, DENV-4, DENV-5), LASV, YSF, ZEBOV, JEV, rhinovirus, coxsackievirus, NiV, CMV, HHV-6, and the like); (ii) those that detect and/or measure a polypeptide encoded by a virus (e.g., for HIV: p24, gp120, reverse transcriptase), using, e.g., an immunological assay such as an enzyme-linked immunosorbent assay (ELISA) with an antibody specific for the polypeptide; (iii) and those that detect and/or measure the activity of a polypeptide encoded by a virus (e.g., measuring reverse transcriptase (RT) enzymatic activity). Suitable tests for determining whether the methods of the present disclosure are effective in reducing viral load can also include those that count the number cells, those of the same type as the target cell, in the individual (e.g., CD4⁺ T cell count when the virus is HIV).

A biological sample to be assayed can be a sample obtained from the individual prior to and/or after contacting a target cell of the individual with a subject anti-viral composition (e.g., prior to and/or after administering to an individual a subject anti-viral composition). For example, a biological sample can be collected prior to and after contacting a target cell of the individual with a subject anti-viral composition (e.g., prior to and after administering to an individual a subject anti-viral composition), and the results can be used to determine whether the treatment has been effective. For example, if viral load in a sample collected after treatment is less than viral load in a sample collected prior to treatment, then the treatment can be considered to be effective.

Methods of measuring viral concentration will be known to one of ordinary skill in the art and may include: plaque assay (determine the number of plaque forming units (PFU) in a sample by plating a known volume and/or dilution of sample with susceptible cells); fluorescent focus assay (FFA); protein bases assays (e.g., hemagglutination assay, bicinchoninic acid assay, single radial immunodiffusion assay, and the like); transmission Electron Microscopy (TEM); flow cytometry (e.g., using antibodies and/or probes against viral specific proteins and/or nucleic acids); etc.

In some embodiments, a subject method of treating an individual to inhibit the activity of a virus involves: a) contacting a target cell of the individual with an effective amount of an anti-viral composition comprising an anti-viral agent, where an effective amount of an anti-viral agent that inhibits virus function (i.e., viral activity). Viral activity can be monitored by assaying/detecting/measuring any of the steps involved in viral replication (e.g., attachment, nucleic acid entry into the cell, integration, transcription, exit by lysis and/or budding, and/or infectious virus particle formation). In addition, the virus function can be selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. Administering to the individual an effective amount of anti-viral agent can reduce viral activity in an virus-infected cell; and can result in one or both of: a reduction of viral load in the individual; and an increase in the number of target cells (e.g., CD4⁺ T cells in the case of HIV) in the individual.

In some embodiments, the amount of time between (a) initial contact with the anti-viral composition, and (b) contact with (or suspected potential contact with) a virus, is up to 72 hours (e.g., up to 66 hours, up to 60 hours, up to 54 hours, up to 48 hours, up to 42 hours, up to 36 hours, up to 30 hours, up to 24 hours, up to 20 hours, up to 16 hours, up to 12 hours, up to 8 hours, up to 6 hours, up to 5 hours, up to 4 hours, up to 3 hours, up to 2 hours, or up to 1 hour). In some embodiments, the amount of time between (a) initial contact with the anti-viral composition, and (b) contact with (or suspected potential contact with) a virus, is in a range of from 1 hour to 72 hours (e.g., from 1 hour to 66 hours, from 1 hour to 60 hours, from 1 hour to 54 hours, from 1 hour to 48 hours, from 2 hours to 66 hours, from 2 hours to 60 hours, from 2 hours to 54 hours, from 2 hours to 48 hours, from 4 hours to 66 hours, from 4 hours to 60 hours, from 4 hours to 54 hours, from 4 hours to 48 hours, from 6 hours to 66 hours, from 6 hours to 60 hours, from 6 hours to 54 hours, or from 6 hours to 48 hours).

In other words, in some embodiments, a target cell is contacted with an anti-viral composition up to 72 hours prior to contact with (or suspected potential contact with) a virus (e.g., up to 66 hours, up to 60 hours, up to 54 hours, up to 48 hours, up to 42 hours, up to 36 hours, up to 30 hours, up to 24 hours, up to 20 hours, up to 16 hours, up to 12 hours, up to 8 hours, up to 6 hours, up to 5 hours, up to 4 hours, up to 3 hours, up to 2 hours, or up to 1 hour prior to contact). In some embodiments, a target cell is contacted with an anti-viral composition in a range of from 1 hour to 72 hours prior to contact with (or suspected potential contact with) a virus (e.g., from 1 hour to 66 hours, from 1 hour to 60 hours, from 1 hour to 54 hours, from 1 hour to 48 hours, from 2 hours to 66 hours, from 2 hours to 60 hours, from 2 hours to 54 hours, from 2 hours to 48 hours, from 4 hours to 66 hours, from 4 hours to 60 hours, from 4 hours to 54 hours, from 4 hours to 48 hours, from 6 hours to 66 hours, from 6 hours to 60 hours, from 6 hours to 54 hours, or from 6 hours to 48 hours prior to contact).

In some embodiments, the target cell is contacted with the anti-viral composition at the same time that the cell is contacted with a virus.

In some embodiments, the target cell is contacted with the anti-viral composition (e.g., an anti-viral agent is administered to an individual) up to 72 hours after being contacted with a virus (or after suspected contact with a virus) (e.g., up to 60 hours, up to 48 hours, up to 42 hours, up to 36 hours, up to 30 hours, up to 24 hours, up to 18 hours, up to 15 hours, up to 12 hours, up to 10 hours, up to 9 hours, up to 8 hours, up to 7 hours, up to 6 hours, up to 5 hours, up to 4 hours, up to 3 hours, up to 2 hours, or up to 1 hour after being contacted). In some embodiments, the target cell is contacted with the anti-viral composition in a range of from 10 minutes to 72 hours after being contacted with a virus (or after suspected contact with the virus) (e.g., from 10 minutes to 60 hours, from 10 minutes to 48 hours, from 10 minutes to 42 hours, from 10 minutes to 36 hours, from 10 minutes to 30 hours, from 10 minutes to 24 hours, from 10 minutes to 18 hours, from 10 minutes to 15 hours, from 10 minutes to 12 hours, from 10 minutes to 10 hours, from 10 minutes to 9 hours, from 10 minutes to 8 hours, from 10 minutes to 7 hours, from 10 minutes to 6 hours, from 10 minutes to 5 hours, from 10 minutes to 4 hours, from 10 minutes to 3 hours, from 10 minutes to 2 hours, from 30 minutes to 60 hours, from 30 minutes to 48 hours, from 30 minutes to 42 hours, from 30 minutes to 36 hours, from 30 minutes to 30 hours, from 30 minutes to 24 hours, from 30 minutes to 18 hours, from 30 minutes to 15 hours, from 30 minutes to 12 hours, from 30 minutes to 10 hours, from 30 minutes to 9 hours, from 30 minutes to 8 hours, from 30 minutes to 7 hours, from 30 minutes to 6 hours, from 30 minutes to 5 hours, from 30 minutes to 4 hours, from 30 minutes to 3 hours, from 30 minutes to 2 hours, from 1 hour to 60 hours, from 1 hour to 48 hours, from 1 hour to 42 hours, from 1 hour to 36 hours, from 1 hour to 30 hours, from 1 hour to 24 hours, from 1 hour to 18 hours, from 1 hour to 15 hours, from 1 hour to 12 hours, from 1 hour to 10 hours, from 1 hour to 9 hours, from 1 hour to 8 hours, from 1 hour to 7 hours, from 1 hour to 6 hours, from 1 hour to 5 hours, from 1 hour to 4 hours, from 1 hour to 3 hours, from 1 hour to 2 hours, from 2 hours to 60 hours, from 2 hours to 48 hours, from 2 hours to 42 hours, from 2 hours to 36 hours, from 2 hours to 30 hours, from 2 hours to 24 hours, from 2 hours to 18 hours, from 2 hours to 15 hours, from 2 hours to 12 hours, from 2 hours to 10 hours, from 2 hours to 9 hours, from 2 hours to 8 hours, from 2 hours to 7 hours, from 2 hours to 6 hours, from 2 hours to 5 hours, from 2 hours to 4 hours, or from 2 hours to 3 hours after being contacted).

In some embodiments, the target cell is contacted with the anti-viral composition (e.g., in a single administration) for a period of time of up to 72 hours (e.g., up to 66 hours, up to 60 hours, up to 54 hours, up to 48 hours, up to 42 hours, up to 36 hours, up to 30 hours, up to 24 hours, up to 20 hours, up to 16 hours, up to 12 hours, up to 8 hours, up to 6 hours, up to 5 hours, up to 4 hours, up to 3 hours, up to 2 hours, or up to 1 hour). In some embodiments, the target cell is contacted with the anti-viral composition for a period of time in a range of from 1 hour to 72 hours (e.g., from 1 hour to 66 hours, from 1 hour to 60 hours, from 1 hour to 54 hours, from 1 hour to 48 hours, from 2 hours to 66 hours, from 2 hours to 60 hours, from 2 hours to 54 hours, from 2 hours to 48 hours, from 4 hours to 66 hours, from 4 hours to 60 hours, from 4 hours to 54 hours, from 4 hours to 48 hours, from 6 hours to 66 hours, from 6 hours to 60 hours, from 6 hours to 54 hours, or from 6 hours to 48 hours).

In some embodiments, anti-viral agent is administered in combination therapy with a known inhibitor of the virus. For example, where the virus is HIV, anti-viral agent can be administered in combination therapy with a known inhibitor of HIV: 1) one or more nucleoside reverse transcriptase inhibitors (e.g., Combivir, Epivir, Hivid, Retrovir, Videx, Zerit, Ziagen, etc.); 2) one or more non-nucleoside reverse transcriptase inhibitors (e.g., Rescriptor, Sustiva, Viramune, etc.); 3) one or more protease inhibitors (e.g., Agenerase, Crixivan, Fortovase, Invirase, Kaletra, Norvir, Viracept, etc.); 4) anti-HIV agent such as a protease inhibitor and a nucleoside reverse transcriptase inhibitor; 5) anti-HIV agent such as a protease inhibitor, a nucleoside reverse transcriptase inhibitor, and a non-nucleoside reverse transcriptase inhibitor; 6) anti-HIV agent such as a protease inhibitor and a non-nucleoside reverse transcriptase inhibitor. Other combinations of an effective amount of anti-viral agent with one or more anti-HIV agents, such as one or more of a protease inhibitor, a nucleoside reverse transcriptase inhibitor, and a non-nucleoside reverse transcriptase inhibitor, are contemplated.

In some embodiments, an anti-viral agent (e.g., sCD137, FAM3C, or a combination thereof) is co-administered with another anti-viral agent known by one of ordinary skill in the art as appropriate (e.g., co-administer a subject anti-viral agent (sCD137 and/or FAM3C) with an antibody against Ebola virus when ebola virus is the subject virus; co-administer a subject anti-viral agent (sCD137 and/or FAM3C) with an antibody against hendra virus when hendra virus is the subject virus; etc.).

### Formulations, Dosages, and Routes of Administration

In general, an active agent (e.g., an anti-viral agent) is prepared in a pharmaceutically acceptable composition(s) for delivery to a host. In the context of reducing viral activity, the terms “active agent,” “drug,” “agent,” “therapeutic agent,” and the like are used interchangeably herein to refer to an anti-viral agent (e.g., a sCD137 polypeptide, a FAM3C polypeptide, a nucleic acid comprising a nucleotide sequence encoding a sCD137 polypeptide, a nucleic acid comprising a nucleotide sequence encoding a FAM3C polypeptide, or a combination thereof).

Pharmaceutically acceptable carriers preferred for use with active agents (and optionally one or more additional therapeutic agent) may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising an active agent (and optionally one or more additional therapeutic agent) may also be lyophilized using means well known in the art, for subsequent reconstitution and use according to the present disclosure.

### Formulations

An anti-viral agent is administered to an individual in need thereof in a formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. For the purposes of the following description of formulations, the term “active agent” includes an anti-viral agent as described above, and optionally one or more additional therapeutic agent.

In a subject method, an active agent may be administered to the host using any convenient means capable of resulting in the desired degree of reduction of viral activity. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host.

Other modes of administration will also find use with a method of the present disclosure. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

Although the dosage used will vary depending on the clinical goals to be achieved, a suitable dosage range of an active agent is one which provides up to about 1 mg to about 1000 mg, e.g., from about 0.25 mg to about 0.5 mg, from about 0.5 mg to about 1 mg, from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, or from about 500 mg to about 1000 mg of an active agent can be administered in a single dose.

In some embodiments, a suitable dose of an antiviral agent (e.g., a sCD137 polypeptide or a FAM3C polypeptide) is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Where two different active agents are administered, a first active agent and a second active agent can be administered in separate formulations. A first active agent and a second active agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, transdermal, subcutaneous, intradermal, topical application, intravenous, vaginal, nasal, and other parenteral routes of administration. In some embodiments, an active agent is administered via an intravaginal route of administration. In other embodiments, an active agent is administered via an intrarectal route of administration. Routes of administration may be combined, if desired, or adjusted depending upon the agent and/or the desired effect. The composition can be administered in a single dose or in multiple doses.

An active agent can be administered to a host using any available conventional methods and routes suitable for delivery of conventional drugs, including systemic or localized routes. In general, routes of administration contemplated by the present disclosure include, but are not necessarily limited to, enteral, parenteral, or inhalational routes.

Parenteral routes of administration other than inhalation administration include, but are not necessarily limited to, topical, vaginal, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

An active agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as the number of viral particles per unit blood. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, and primates (e.g., humans, chimpanzees, and monkeys), that are susceptible to viral infection. In many embodiments, the hosts will be humans.

### Kits, Containers, Devices, Delivery Systems

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present disclosure provides a delivery system comprising an active agent that inhibits viral activity. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present disclosure provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the present disclosure provides an injection delivery device that is pre-loaded with a formulation comprising an effective amount of anti-viral agent. For example, a subject delivery device comprises an injection device pre-loaded with a single dose of anti-viral agent. A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present disclosure provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909. If a tampon or tampon-like device is used, there are numerous methods by which an active agent can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon, for example, by dissolving the drug in a pharmaceutically acceptable carrier and absorbing the drug solution into the tampon fibers. The drug can also be dissolved in a coating material which is applied to the tip of the tampon. Alternatively, the drug can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no drug. Optionally, the third ring can contain a second drug for a dual release ring. The drug can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used. These systems have been described extensively in the literature.

Bioadhesive microparticles constitute still another drug delivery system suitable for use in a formulation or a method of the present disclosure. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present disclosure will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Combination Therapy

In some embodiments, anti-viral agent is administered in combination therapy with one or more additional therapeutic agents. Suitable additional therapeutic agents include agents that inhibit one or more functions of a virus; agents that treat or ameliorate a symptom of a virus infection; agents that treat an infection that occurs secondary to a virus infection; and the like.

A subject combination therapy can involve: a) administration of a sCD137 polypeptide or a FAM3C polypeptide, and at least one additional therapeutic agent at the same time, in the same formulation or in separate formulations; b) administration of at least one additional therapeutic agent within about 5 minutes to about 4 weeks of administration of a sCD137 polypeptide or a FAM3C polypeptide, e.g., administration of at least one additional therapeutic agent within about 5 minutes to about 15 minutes, within about 15 minutes to about 30 minutes, within about 30 minutes to about 60 minutes, within about 1 hour to about 2 hours, within about 2 hours to about 4 hours, within about 4 hours to about 8 hours, within about 8 hours to about 12 hours, within about 12 hours to about 24 hours, within about 24 hours to about 2 days, within about 2 days to about 4 days, within about 4 days to about 7 days, within about 1 week to about 2 weeks, or within about 2 weeks to about 4 weeks of administration of a sCD137 polypeptide or a FAM3C polypeptide.

In some embodiments, the at least one additional therapeutic agent is co-formulated with a sCD137 polypeptide or a FAM3C polypeptide. In other embodiments, the at least one additional therapeutic agent and a sCD137 polypeptide or a FAM3C polypeptide are separately formulated.

In some embodiments, a sCD137 polypeptide or a FAM3C polypeptide is administered for a first period of time, and an at least one additional therapeutic agent is administered for a second period of time, where the first period of time and the second period of time are overlapping. For example, in some embodiments, a sCD137 polypeptide or a FAM3C polypeptide is administered for a first period of time, and at least one additional therapeutic agent is administered for a second period of time, where the second period of time begins before the end of the first period of time. In some embodiments, a sCD137 polypeptide or a FAM3C polypeptide is administered for a first period of time, and an at least one additional therapeutic agent is administered for a second period of time, where the first period of time begins before the end of the second period of time. In some embodiments, a sCD137 polypeptide or a FAM3C polypeptide is administered for a first period of time, and an at least one additional therapeutic agent is administered for a second period of time, where the first period of time begins before the beginning of the second period of time, and ends after the end of the second period of time.

In some embodiments, an effective amount of a sCD137 polypeptide or a FAM3C polypeptide and an at least one additional therapeutic agent are synergistic amounts. As used herein, a “synergistic combination” or a “synergistic amount” of a sCD137 polypeptide or a FAM3C polypeptide and an additional (e.g., a second) therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the sCD137 polypeptide or the FAM3C polypeptide when administered at that same dosage as a monotherapy and (ii) the therapeutic or prophylactic benefit of the additional therapeutic agent when administered at the same dosage as a monotherapy.

### Additional Therapeutic Agents

For example, where a subject method inhibits viral activity of an immunodeficiency virus (e.g., HIV), suitable additional therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™) stavudine (d4T, Zerit™), zalcitabine (dideoxycytosine, ddC, Hivid™), nevirapine (Viramune™) lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, anti-viral agent is administered in combination therapy with one, two, or more than two, anti-HIV agents. For example, anti-viral agent can be administered in combination therapy with one, two, or three nucleoside reverse transcriptase inhibitors (e.g., Combivir, Epivir, Hivid, Retrovir, Videx, Zerit, Ziagen, etc.). Anti-viral agent can be administered in combination therapy with one or two non-nucleoside reverse transcriptase inhibitors (e.g., Rescriptor, Sustiva, Viramune, etc.). Anti-viral agent can be administered in combination therapy with one or two protease inhibitors (e.g., Agenerase, Crixivan, Fortovase, Invirase, Kaletra, Norvir, Viracept, etc.). Anti-viral agent can be administered in combination therapy with a protease inhibitor and a nucleoside reverse transcriptase inhibitor. Anti-viral agent can be administered in combination therapy with a protease inhibitor, a nucleoside reverse transcriptase inhibitor, and a non-nucleoside reverse transcriptase inhibitor. Anti-viral agent can be administered in combination therapy with a protease inhibitor and a non-nucleoside reverse transcriptase inhibitor. Other combinations of anti-viral agent with one or more of a protease inhibitor, a nucleoside reverse transcriptase inhibitor, and a non-nucleoside reverse transcriptase inhibitor are contemplated.

In some embodiments, a subject treatment method involves administering: a) anti-viral agent; and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) anti-viral agent; and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea.

Nucleoside reverse transcriptase inhibitors include, but are not limited to, abacavir (ABC; ZIAGEN™), didanosine (dideoxyinosine (ddI); VIDEX™), lamivudine (3TC; EPIVIR™), stavudine (d4T; ZERIT™, ZERIT XR™), zalcitabine (dideoxycytidine (ddC); HIVID™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRIPTOR™), and efavirenz (SUSTIVA™).

Protease inhibitors (PIs) for treating HIV infection include amprenavir (AGENERASE™) saquinavir mesylate (FORTOVASE™, INVIRASE™), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™).

Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy)lup-20(29)-en-28-oic acid); and Vivecon (MPC9055).

In some embodiments, a subject treatment method involves administering: a) anti-viral agent; and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, R00334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SP01A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, INCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS119, ALG 889, and PA-1050040 (PA-040); (13) any combinations or mixtures of the above.

As further examples, in some embodiments, a subject treatment method involves administering: a) anti-viral agent; and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily; iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)-N- [(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)-N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) in an amount of 133 mg twice daily; vi) fosamprenavir (Lexiva; {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily); vii) ritonavir (Norvir) in an amount of 600 mg twice daily; viii) nelfinavir (Viracept; (3S,4aS,8aS)-N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide; SEQ ID NO:16) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[(2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; (1S,4R)-4-{[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily; xiv) Trizivir in an amount of 150 mg 3TC and 300 mg ABV and 300 mg AZT twice daily; xv) Truvada in an amount of 200 mg emtricitabine and 300 mg tenofovir (({[(2R)-1-(6-amino-9H-purin-9-yl)propan-2-yl]oxy}methyl)phosphonic acid) once daily; xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

Suitable additional therapeutic agents, e.g., for the treatment of an HSV-1 or an HSV-2 infection include, but are not limited to, acyclovir (Zovirax), valganciclovir, famciclovir, valacyclovir (Valtrex), ganciclovir (Cytovene), cidofovir (Vistide), antisense oligonucleotide fomivirsen (Vitravene), foscarnet (Foscavir), penciclovir, idoxuridine, vidarabine, and trifluridine. In some embodiments, the at least one additional therapeutic agent is an interferon (e.g., interferon-alpha, interferon-beta, interferon-gamma, interferon-lambda, interferon-tau, interferon-omega, etc.).

Acyclovir is a purine nucleoside analog that can be used in a subject combination therapy for treating HSV-1, HSV-2, VZV, or EBV infection. Valacyclovir can be used in a subject combination therapy for treating HSV-1, HSV-2, VZV, or EBV infection. Cidofovir is a nucleotide analog that can be used in a subject combination therapy for treating HSV-1, HSV-2, VZV, EBV, or KSHV infection. Famciclovir is a prodrug that can be used in a subject combination therapy for treating HSV-1, HSV-2, or VZV infection. Foscarnet is an organic analog of inorganic pyrophosphate that can be used in a subject combination therapy for treating EBV, KSHV, HSV, or VZV infection. Ganciclovir is a nucleoside analog of 2′-deoxyguanosine that can be used in a subject combination therapy for treating any human herpesvirus (HHV) infection. Valganciclovir is an orally bioavailable form of ganciclovir that can be used in a subject combination therapy for treating any HHV infection. Idoxuridine can be used topically in a subject combination therapy to treat herpes simplex keratoconjunctivitis. Penciclovir is a phosphorylated guanosine analog that can be applied topically in a subject combination therapy to treat recurrent herpes labialis (e.g., caused by HSV-1 or HSV-2). Trifluridine is a thymine analog that can be used in a subject combination therapy for treating primary keratoconjunctivitis and recurrent keratitis or ulceration caused by HSV-1 and HSV-2. Vidarabine is an adenine arabinoside that can be used in a subject combination therapy for treating HSV-1 or HSV-2 infection.

Suitable routes of administration of the aforementioned additional therapeutic agents are known in the art. For example, ganciclovir is available as an oral formulation; cidofovir and fomivirsen are approved for topical application against retinitis in AIDS patients; and foscarnet is formulated for use by an intravenous route.

In some embodiments, the use of combination therapy to treat a viral infection, involves administering i) an effective amount of a sCD137 polypeptide or a FAM3C polypeptide; and ii) at least one additional therapeutic agent, where the at least one additional is an interferon. Suitable interferons include, e.g., interferon-alpha (IFN-α), interferon-beta (IFN-β), interferon-gamma (IFN-γ), interferon-lambda (IFN-λ), IFN-tau, IFN-ω, etc. In some cases, the second therapeutic agent is IFN-α. Any known IFN-α can be used in a subject combination therapy. The term “IFN-α” includes biologically active IFN-α, where biologically active IFN-α includes naturally occurring IFN-α; synthetic IFN-α; derivatized IFN-α (e.g., PEGylated IFN-α, glycosylated IFN-α, and the like); glycosylated IFN-α; IFN-α derivatized with poly(ethylene glycol) (“PEGylated IFN-α”); and analogs of naturally occurring or synthetic IFN-α.

In some embodiments, the at least one additional therapeutic agent is ribavirin or a ribavirin derivative. Ribavirin, 1-β-3-D-ribofuranosyl-1H-1,2,4-triazole-3-carboxamide, is described in the Merck Index, compound No. 8199, Eleventh Edition. Its manufacture and formulation is described in U.S. Pat. No. 4,211,771. Also suitable for use are derivatives of ribavirin (see, e.g., U.S. Pat. No. 6,277,830). The ribavirin can be administered orally in capsule or tablet form, or in the same or different administration form and in the same or different route as the sCD137 polypeptide or the FAM3C polypeptide. Other routes/modes of administration of both medicaments are contemplated, such as by nasal spray, transdermally, by suppository, by sustained release dosage form, etc. Any form of administration will work so long as the proper dosages are delivered without destroying the active ingredient.

Ribavirin can be administered in an amount ranging from about 400 mg to about 1200 mg, from about 600 mg to about 1000 mg, or from about 700 to about 900 mg per day. In some embodiments, ribavirin is administered throughout the entire course of sCD137 polypeptide or FAM3C polypeptide therapy. In other embodiments, ribavirin is administered only during the first period of time. In still other embodiments, ribavirin is administered only during the second period of time.

In some embodiments, the at least one additional therapeutic agent is a neuraminidase inhibitor, e.g., where the influenza virus is influenza A or influenza B. Suitable neuraminidase inhibitors include, e.g., oseltamivir (ethyl(3R,4R,5S)-5-amino-4-acetamido-3-(pentan-3-yloxy)cyclohex-1-ene-1-carboxylate; Tamiflu™), zanamivir (2R,3R,4S)-4-[(diaminomethylidene)amino]-3-acetamido-2-[(1R,2R)-1,2,3-trihydroxypropyl]-3,4-dihydro-2H-pyran-6-carboxylic acid; Relenza™), and peramivir (1S,2S,3S,4R)-3-[(1S)-1-acetamido-2-ethyl-butyl]-4-(diaminomethylideneamino)-2-hydroxy-cyclopentane-1-carboxylic acid). In some embodiments, the at least one additional therapeutic agent is an M2 blocker, e.g., blocks a viral ion channel (M2 protein). The antiviral drugs amantadine and rimantadine are M2 blockers, and can be used in subject method of treating an influenza A virus infection.

### Subjects Suitable for Treatment

The methods of the present disclosure are suitable for treating individuals who are infected with a virus, have an increased risk of being infected with a virus, or are suspected of being infected with a virus.

The methods of the present disclosure are suitable for treating individuals who have an HIV infection (e.g., who have been diagnosed as having an HIV infection), and individuals who are at risk of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

In some embodiments, an individual who is suitable for treatment with a subject treatment method is an individual who has not yet been infected with a virus, but who is at greater risk than the general population of becoming infected. Such individuals include, e.g., individuals who are possibly or likely exposed to a virus-infected individual, where such individuals include, e.g., medical personnel, military personnel, prison inmates, and any individual living in a population that includes at least one virus-infected individual.

Individuals who are at greater risk, if infected, of developing complications or experiencing more severe symptoms, than the general population, include, but are not limited to, immunocompromised individuals.

Individuals suitable for treatment with a subject method include, e.g., a human, where the human is immunocompromised. Immunocompromised individuals include, e.g., individuals infected with a human immunodeficiency virus, e.g., where the individual has a lower than normal CD4⁺ T cell count. The normal range of CD4⁺ T cell for humans is from about 600 to about 1500 CD4⁺ T lymphocytes per mm³ blood. Thus, in some embodiments, an immunocompromised individual has a CD4⁺ T cell count that is less than about 600 CD4⁺ T cells per mm³ blood.

Immunocompromised individuals include individuals who are immunocompromised as a result of treatment with a cancer chemotherapeutic agent; and individuals who are immunocompromised as a result of radiation therapy (e.g., for the treatment of a cancer). Immunocompromised individuals include individuals who are immunocompromised due to chronic disease, e.g., cancer, diabetes mellitus, rheumatologic diseases (e.g., systemic lupus erythematosus, etc), immunoglobulin deficiency diseases, and the like. Immunocompromised individuals include transplant recipients (e.g., lung transplant recipients, kidney transplant recipients, bone marrow transplant recipients, etc.). Immunocompromised individuals include individuals who are immunocompromised as a result of taking certain medications such as steroids, chemotherapeutic agents, TNF-α inhibitors, and the like.

Individuals suitable for treatment with a subject method include individuals who are immunosuppressed, e.g., individuals who are undergoing immunosuppressive treatment, where such individuals include, e.g., transplant recipients. Transplant recipients include, e.g., allograft recipients, and the like. Immunosuppressive treatments include, e.g., treatment with FK506.

Individuals suitable for treatment with a subject method include, e.g., a human, where the human is from about one month to about 6 months, from about 6 months to about 1 year, or from about 1 year to about 5 years of age. Individuals suitable for treatment with a subject method include, e.g., a human, where the human is from about 5 years to about 12 years, from about 13 years to about 18 years, or from about 18 years to about 25 years of age. Individuals suitable for treatment with a subject method include, e.g., a human, where the human is from about 25 years to about 50 years, from about 50 years to about 75 years of age, or older than 75 years of age.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

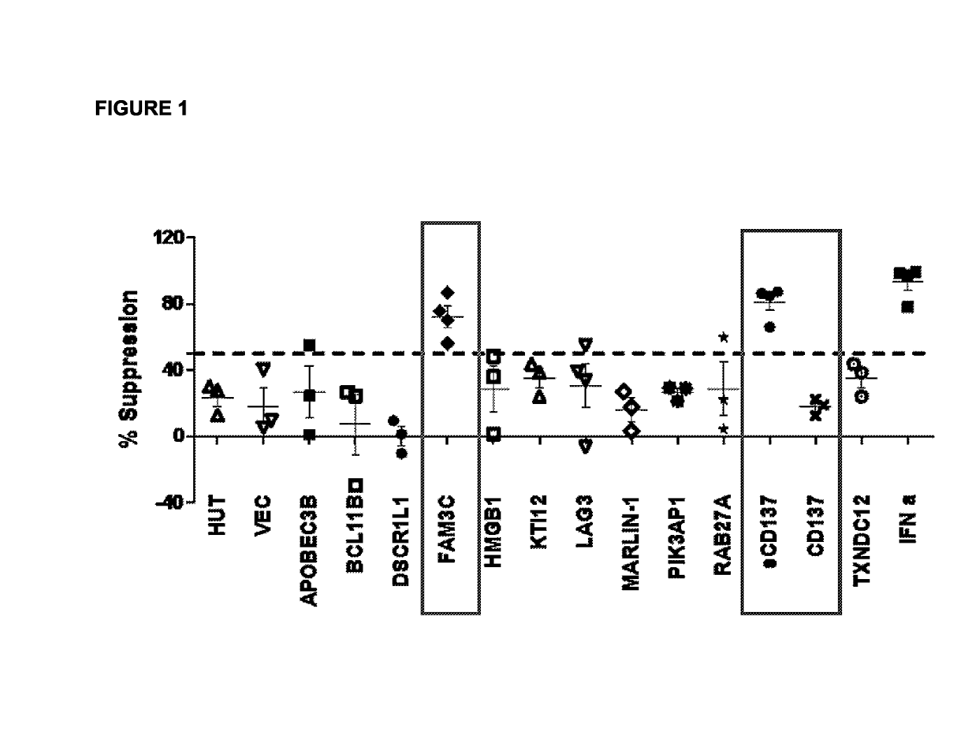

Primary CD8⁺ T cells were obtained from PBMC (peripheral blood mononuclear cells) and were transfected using Neon technology (Liu L et al., Transfection optimization for primary human CD8⁺ cells. J. Immunol. Methods 2011; 372:22-9) with DNA plasmids encoding FAM3C, CD137, or soluble CD137 (sCD137). Two days after transfection, the transfected CD8+ cells were mixed with HIV-infected CD4⁺ cells and virus replication was measured using a reverse transcriptase activity assay (FIG. 1). The percent suppression reflects the extent that the CD8⁺ cells prevented HIV replication in the target CD4⁺ cells. This assay measured CNAR (the CD8⁺ cell non-cytotoxic antiviral response). Only cells transfected with FAM3C and sCD137 suppressed HIV replication.

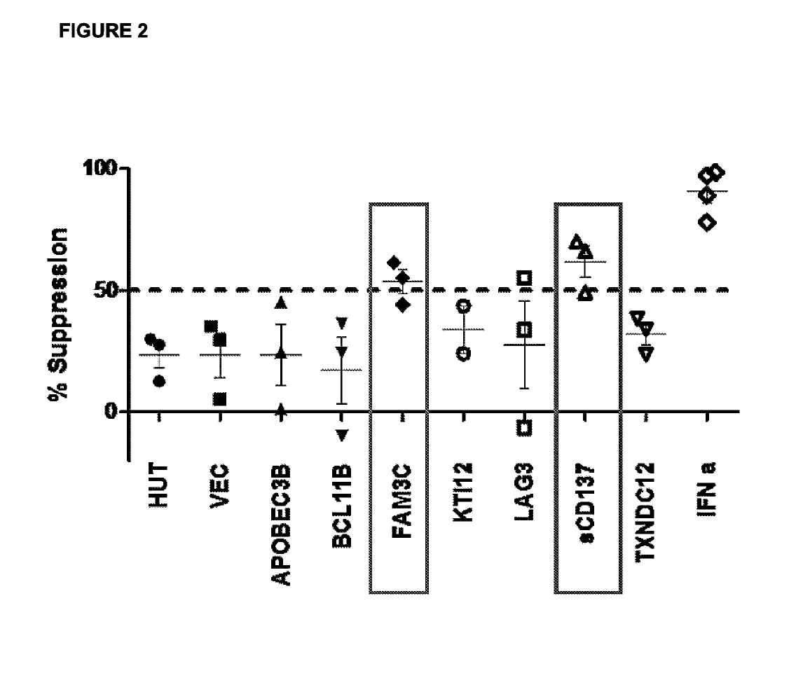

The supernatant (i.e., conditioned media) from transfected CD8⁺ cells showed anti-HIV activity (more than 50% suppression) when added to HIV acutely infected CD4⁺ cells (FIG. 2). Note that interferon-α (IFNα) was used as a positive control that suppresses HIV. Conditioned media from cells transfected with FAM3C or sCD137 (and not full-length CD137) suppressed HIV replication. Only fluids from CD8+ cells transfected with FAM3C and sCD137 suppressed HIV replication.

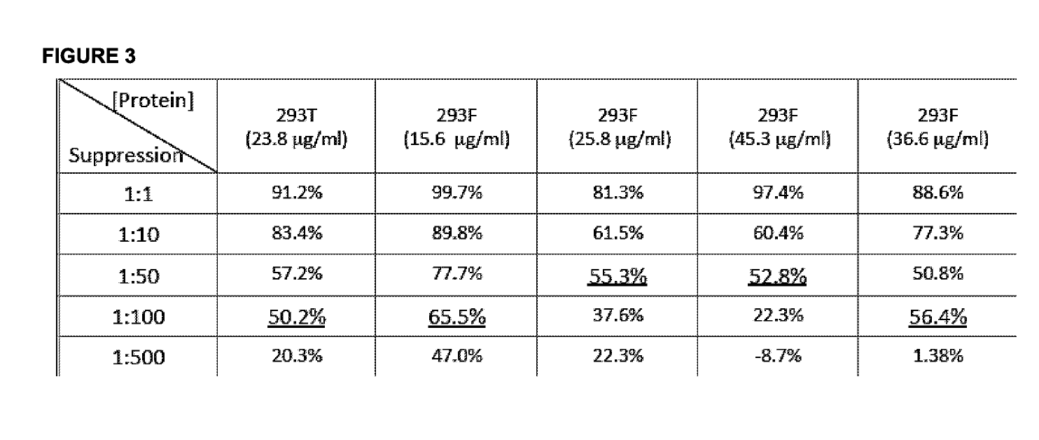

To find the effective dose (ED50) of sCD137 produced by sCD137-transfected HEK (Human Embryonic Kidney) 293 cells, the anti-HIV activity of conditioned media were tested at multiple different concentrations and dilutions of sCD137 (FIG. 3). The average ED50 in HEK293 cell supernatants (i.e., conditioned media) was determined to be 255 ng/ml. In similar experiments with supernatants from sCD137-transfected primary CD8+ cells the supernatant had an average ED50 of 75.8 ng/ml. Thus, expression in the primary CD8+ cell resulted in increased anti-HIV activity.

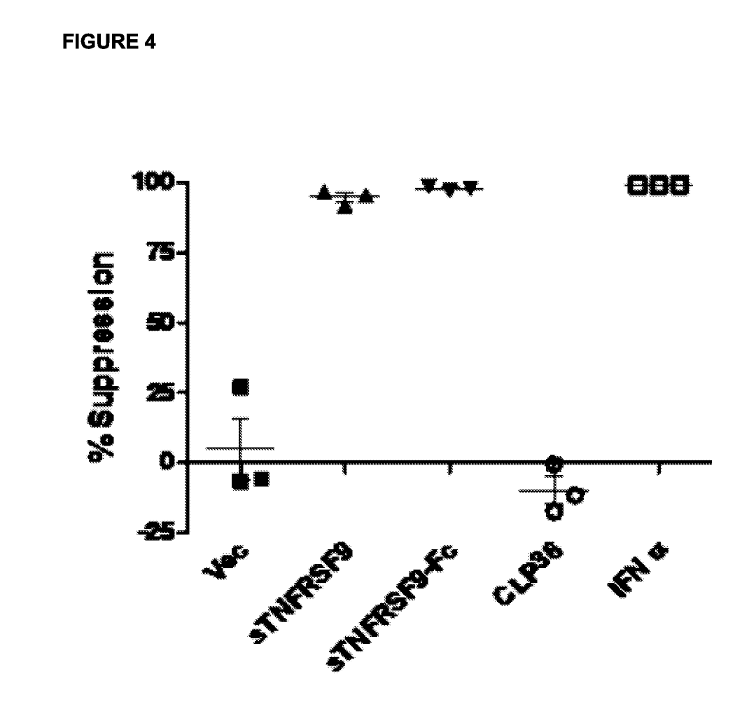

To facilitate purification of sCD137 from conditioned media, a polynucleotide encoding sCD137 was cloned into an expression construct containing an Fc tag. Thus, expression of sCD137 from the expression construct resulted in a sCD137 fusion protein (sCD137 fused to the Fc tag). HEK293 supernatants expressing the sCD137-Fc performed as well as the untagged sCD137 (also called sTNFRSF9), demonstrating that the fusion protein retained its anti-HIV activity (FIG. 4). IFN-α was used as a positive control.

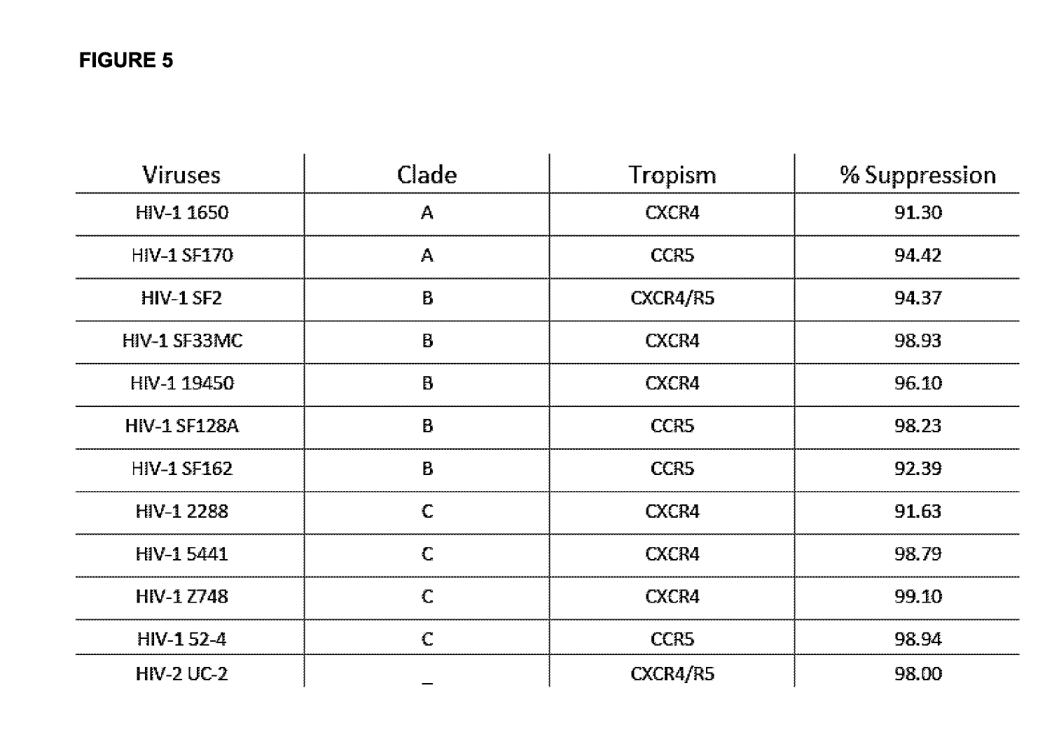

Supernatant from transfected HEK293 cells showed that the sCD137 produced by the transfected cells blocked the activity of a wide variety of HIV viruses, including HIV-1 of clades A, B, and C, as well as HIV-2 (FIG. 5). Note that the tropism of the HIV varied as some types use CXCR4 chemokine co-receptor, others use the CCR5 chemokine co-receptor, and others use both (CXCR4/R5).

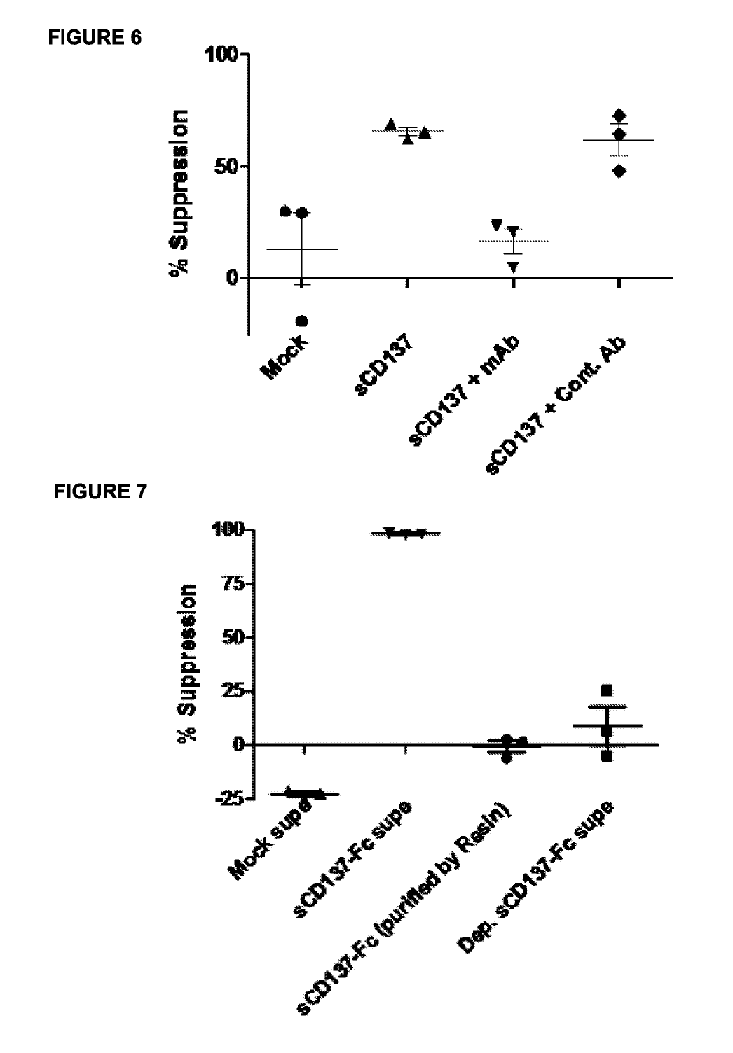

A monoclonal antibody (mAb) against sCD137 was used to neutralize the antiviral effect of supernatant from sCD137-transfected cells when assayed on primary CD4⁺ cells acutely infected with HIV (FIG. 6). Control antibody had no effect. The mock supernatant represents supernatants from HEK293 cells that are transfected with the plasmid vector alone.

When antibodies to sCD137 were added to a culture containing suppressing CD8⁺ cells (not expressing transfected sCD137) mixed with HIV-infected CD4⁺ cells (the CNAR assay), the inhibition by the CD8⁺ cells was not blocked. Thus, although CD8+ cells contain the membrane-bound full CD137 protein, the antibody to the soluble portion of CD137 (sCD137) can only block the soluble version of CD137, which further suggests that the sCD137 is not involved in blocking viral activity in HIV replication during CNAR.

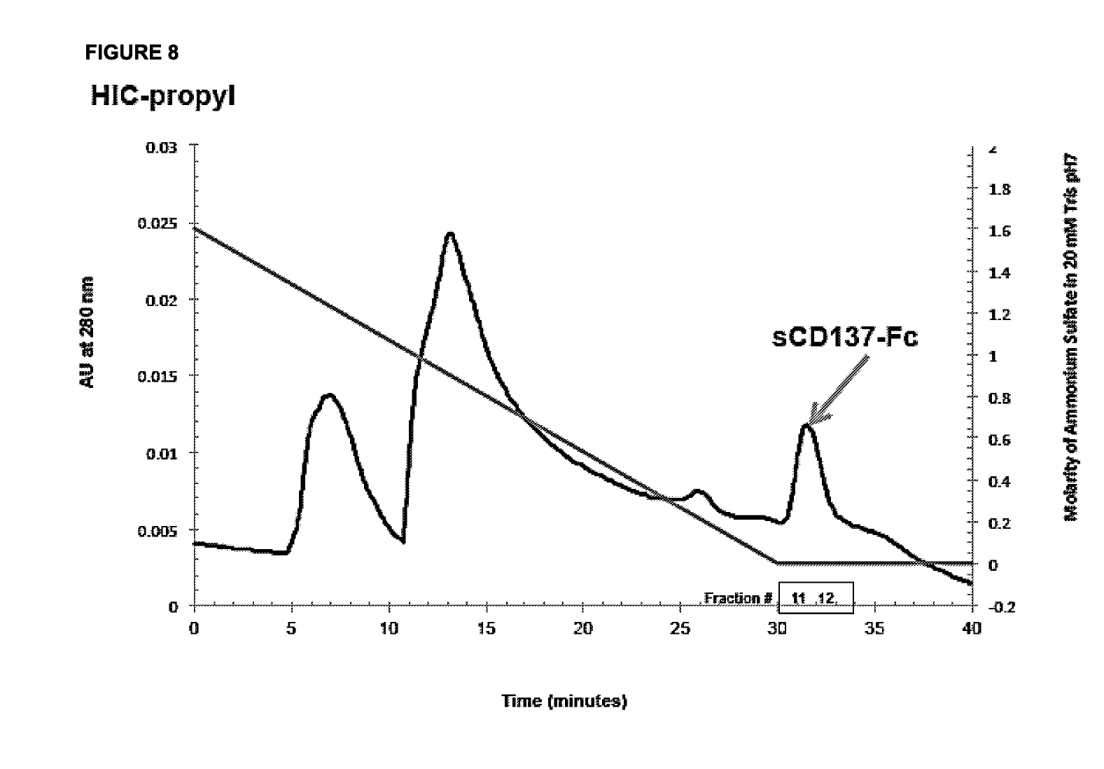

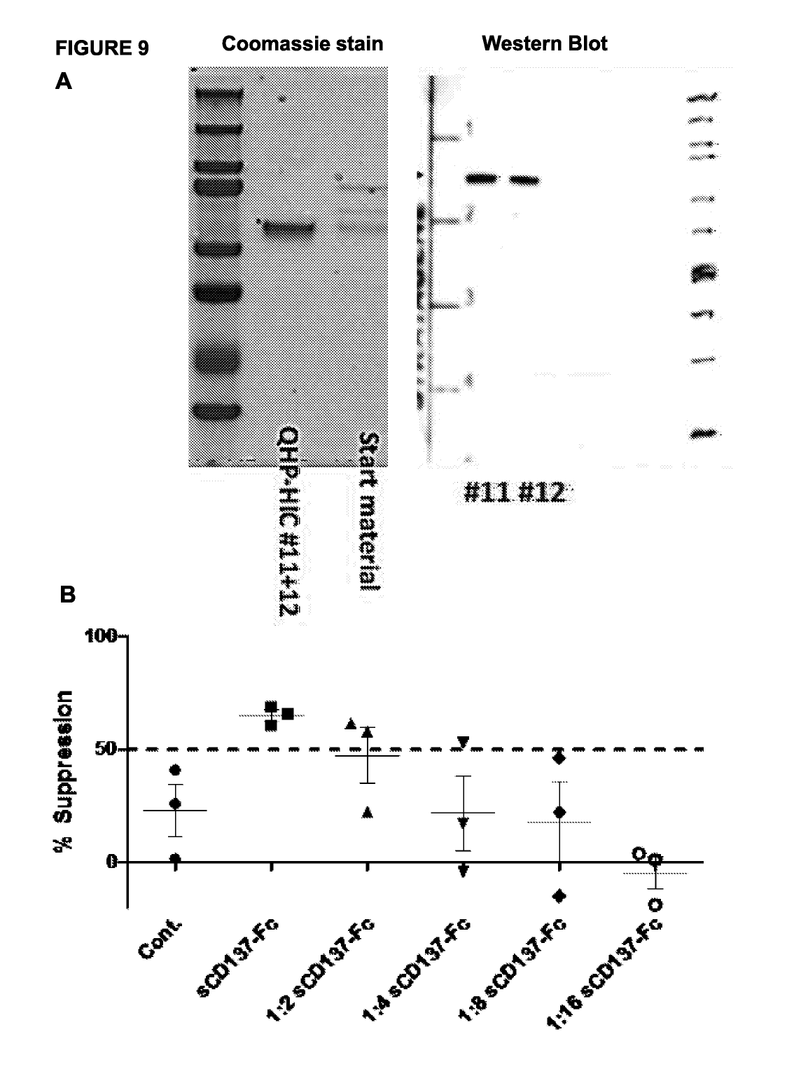

When the sCD137-Fc was removed from supernatant the antiviral activity of sCD137 was also removed (FIG. 7) (Depleted (dep.) sCD137-Fc supernatant (supe)). However, when sCD137-Fc was purified using a resin, it lost anti-HIV activity. To the contrary, when sCD137-Fc was purified using QHP and HIC column chromatography (FIG. 8), it maintained robust anti-HIV activity (FIG. 9B). The sCD137-Fc peak indicated in FIG. 8 was confirmed to be sCD137 via Western blot using an anti-sCD137 antibody (FIG. 9A).

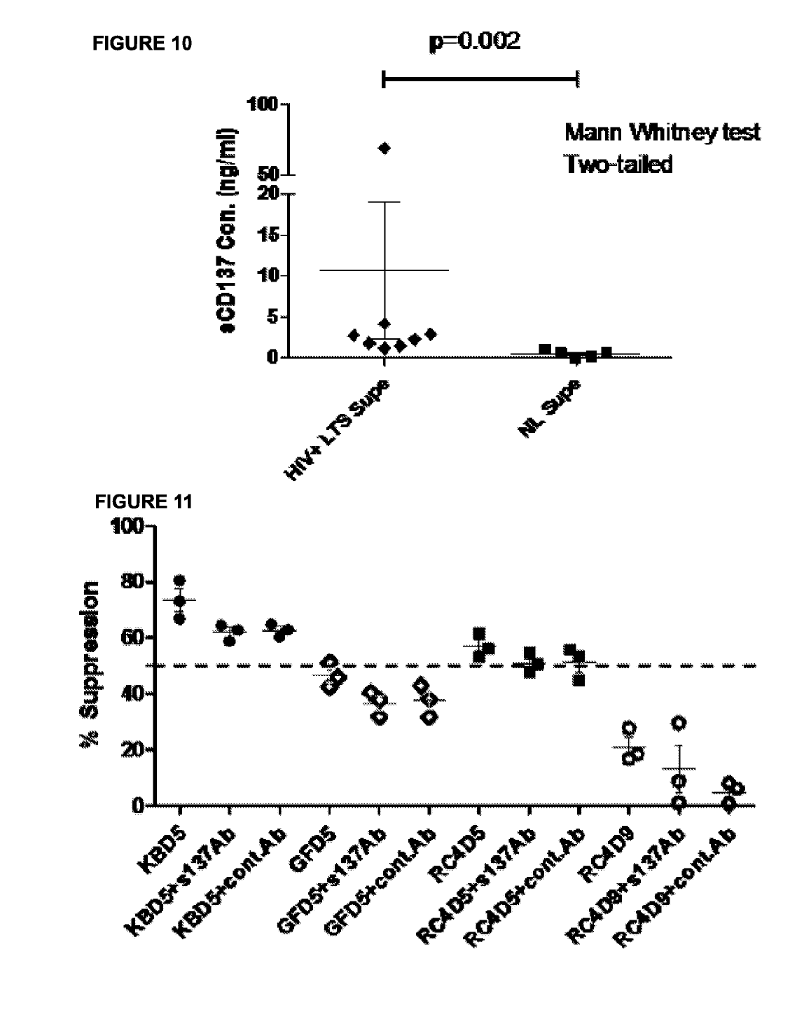

sCD137 levels in CD8⁺ cell supernatants from HIV-infected long-term survivors (LTS) and normal (NL) subjects were measured by an ELISA (FIG. 10). LTS are individuals infected with HIV for more than 10 years, and without therapy have normal CD4+ cell numbers and no symptoms of HIV infection. One LTS had a detectable level of sCD137, but not high enough to show anti-HIV activity. Very low (if any) levels of sCD137 were found in primary CD8+ cells from NL subjects.

Supernatants containing varying levels of CAF (Levy (2003) Trends Immunol. 24:628; Walker et al. (1989) Immunology April; 66(4):628-30) from three subjects were incubated with antibodies to sCD137 to remove any sCD137 that may be present (FIG. 11). Depletion of sCD137 from CAF-positive supernatant did not reduce the antiviral activity, thus indicating that CAF is not sCD137.

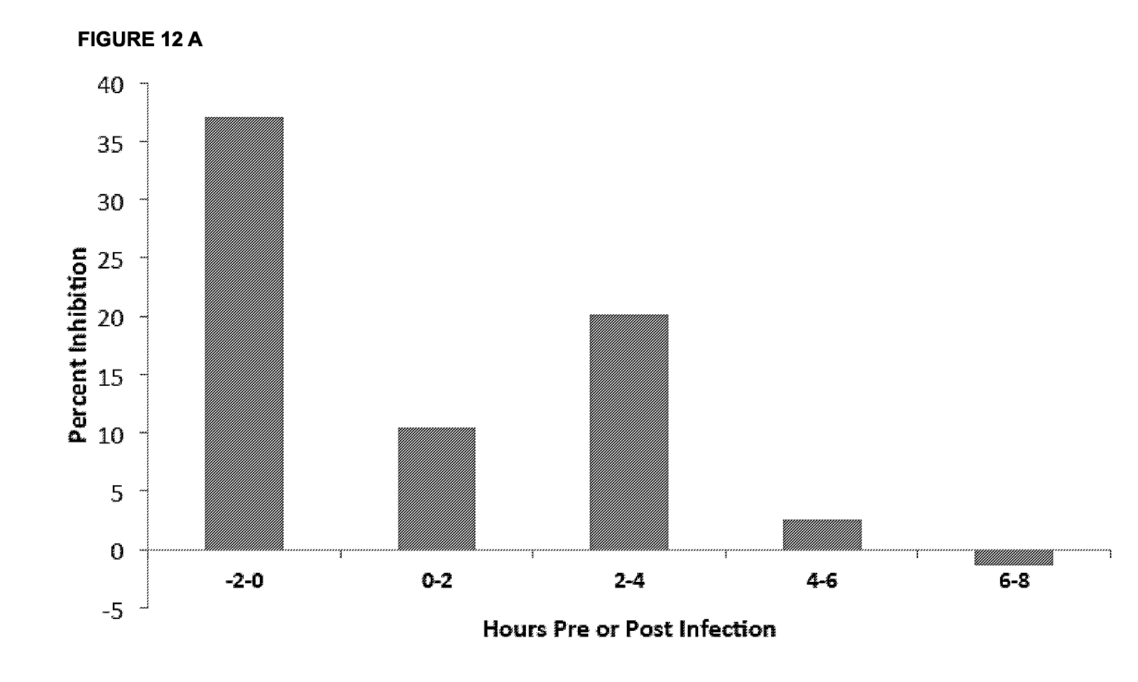

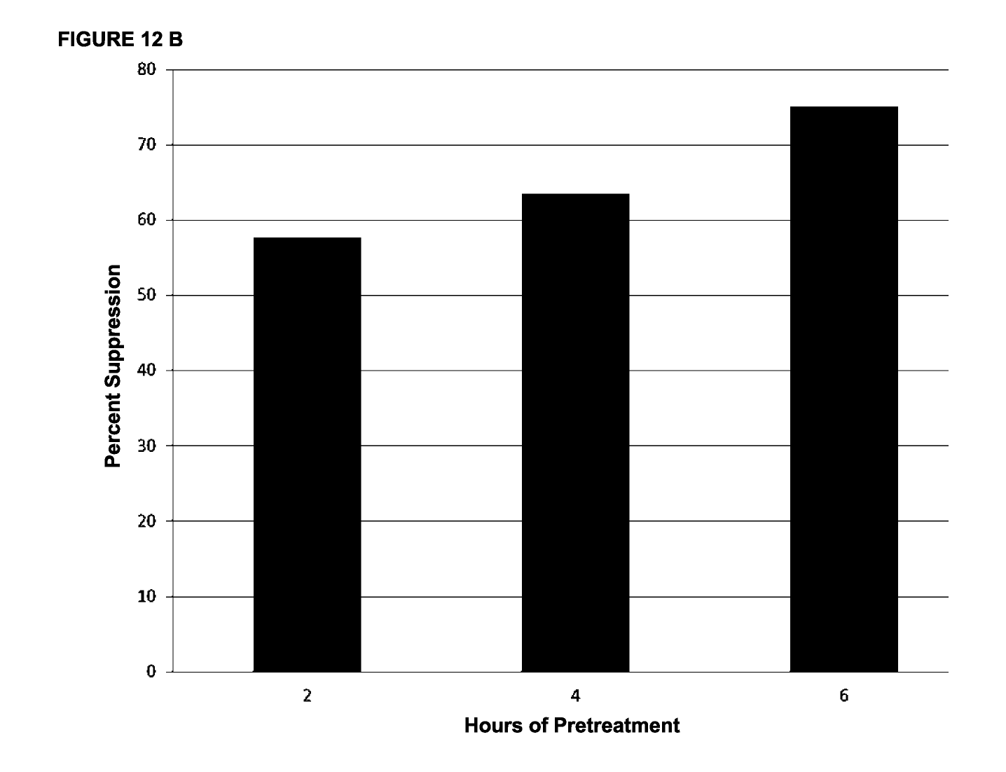

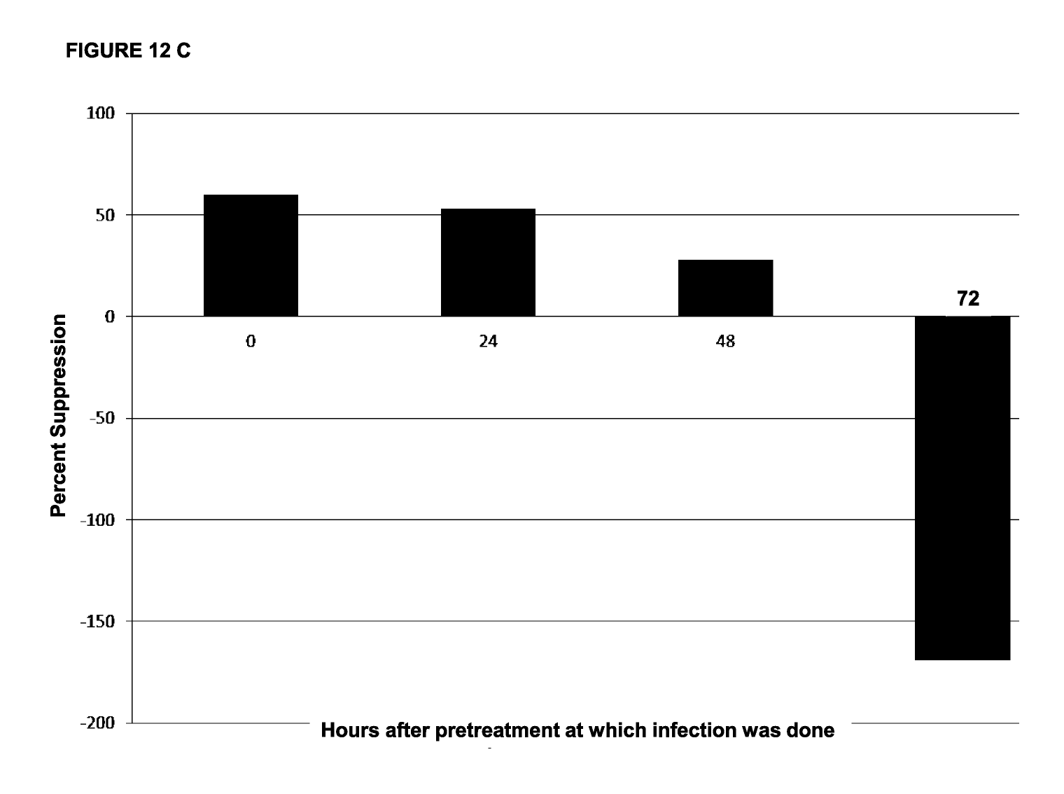

To test for timing of the effect of sCD137 treatment, primary CD4⁺ cells were exposed to sCD137 two hours prior to HIV infection, or at the times indicated following HIV infection (FIG. 12A). Cells were washed before adding HIV. Following HIV infection, sCD137 was added to the culture every 2 hours and washed out after 2 hours as indicated. The antiviral effect was most pronounced when the CD4⁺ cells were treated pre-infection (pre-treated for up to about 2 hours prior to infection) and when treated post-infection (treated after HIV infection) for up to 4 hours following HIV infection.

Pre-treatment for 6-24 hours established a consistent anti-viral state. FIG. 12B shows that increased times of pretreatment of CD4+ cells with purified sCD137 (600 ng/ml) resulted in increased suppression. FIG. 12C shows that an antiviral state was established by the pre-treatment of cells (CD4+ T cells in this particular case) with sCD137 and that the antiviral state lasted for at least 48 hours.

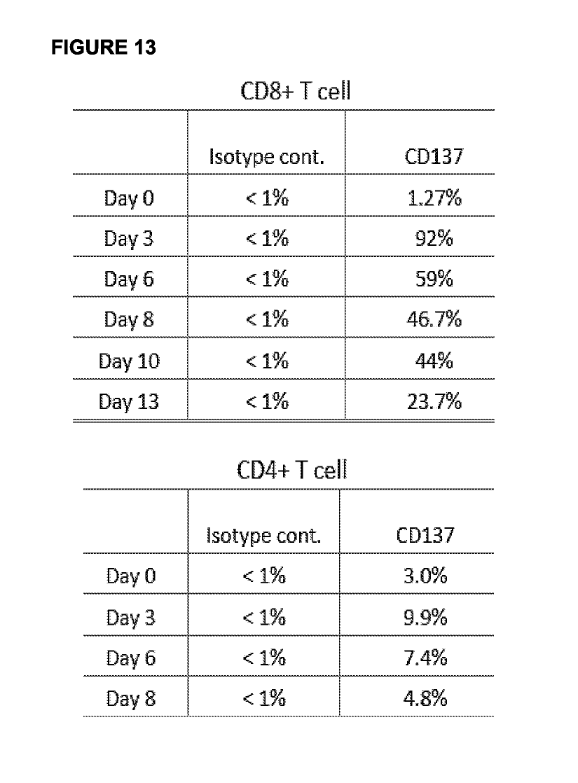

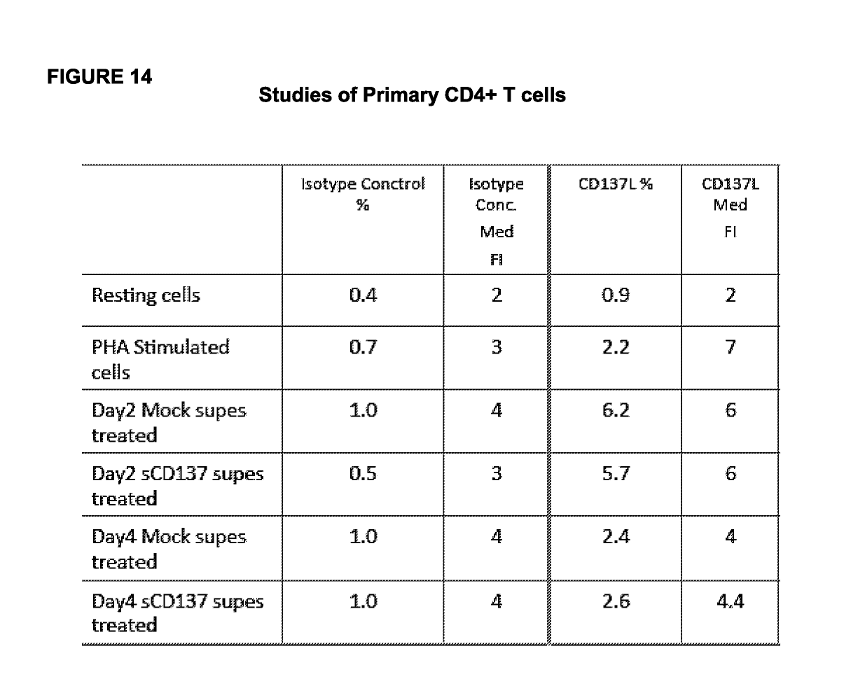

Flow cytometry was used to measure the extent of CD137 expression on CD8+ and CD4+ T cells following activation (FIG. 13). High levels of membrane-bound CD137 are seen on CD8+ cells and lower levels on CD4⁺ T cells. Levels of the reported natural ligand for CD137 (CD137L) was measured on CD4⁺ T cells and very low levels of expression of the ligand was observed (FIG. 14).

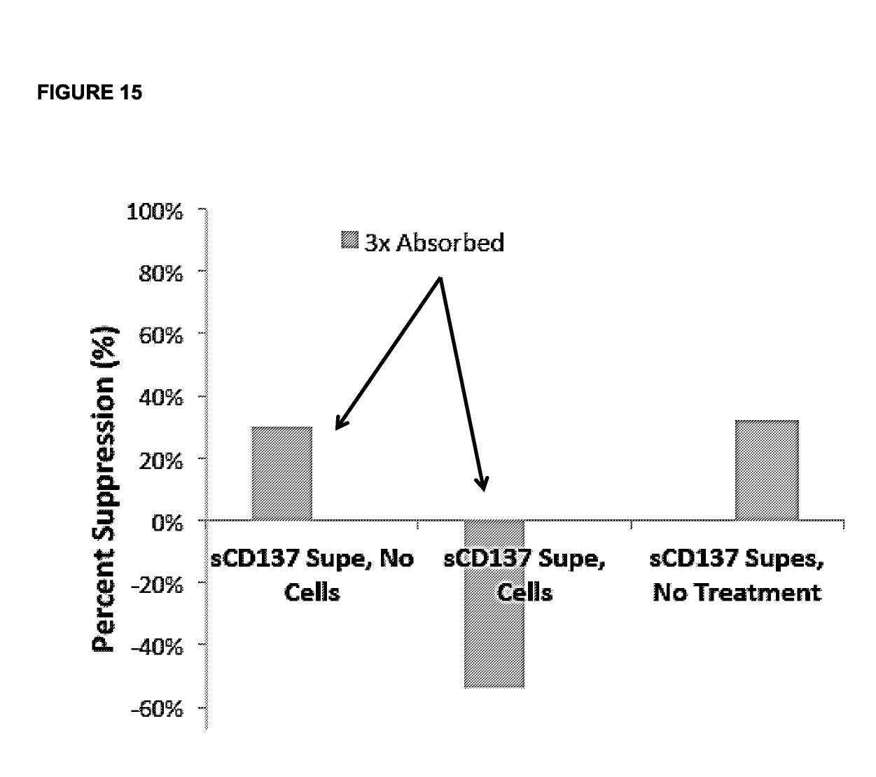

sCD137-containing supernatants diluted 1:25 were incubated both with and without CD4+ cells for 1 hr at 37° C. Cells were spun out and supernatants incubated on another set of naive CD4+. This was repeated 3× for sCD137. Supernatants were then assayed on HIV infected cells for 4 days for anti-HIV activity (FIG. 15). The results demonstrate that primary CD4+ T cells can absorb or remove sCD137 from sCD137-containing supernatants.

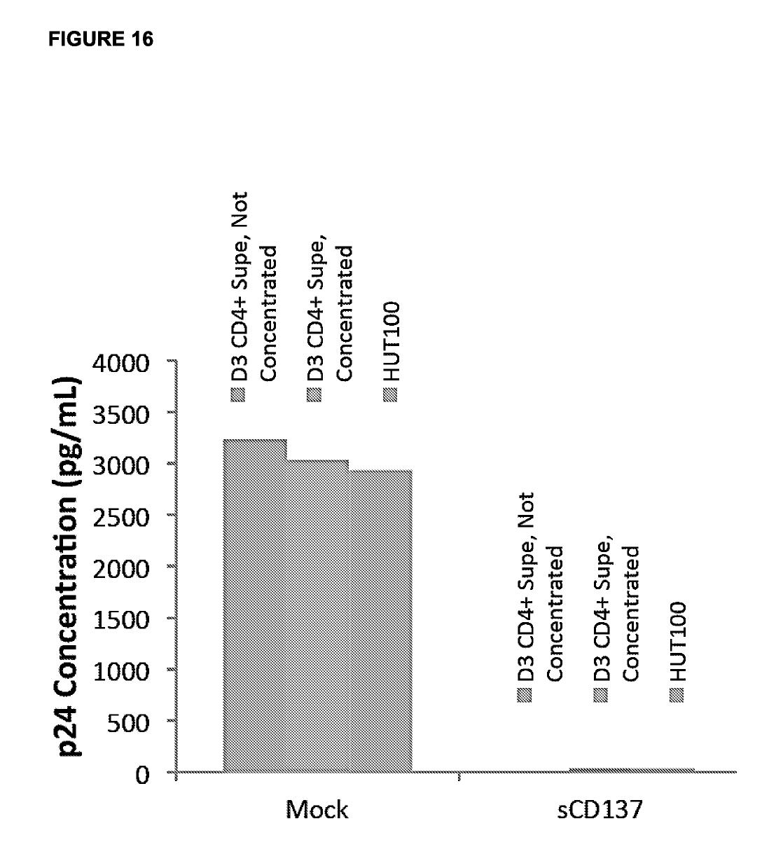

CD4+ cell supernatants were mixed with sCD137-containing supernatants to determine if a secretion by CD4+cells was needed for the antiviral effect of sCD137 (FIG. 16). HIV-1 infected CD4+ cells were cultured for 4 days with different mixtures (CD4+ supernatants (concentrated 10 times or unconcentrated) mixed with sCD137-containing supernatants). The results indicate that a secretion by CD4+cells was not needed for the antiviral effect of sCD137. Concentrated CD4+ cell fluids showed no effect on the activity of sCD137.

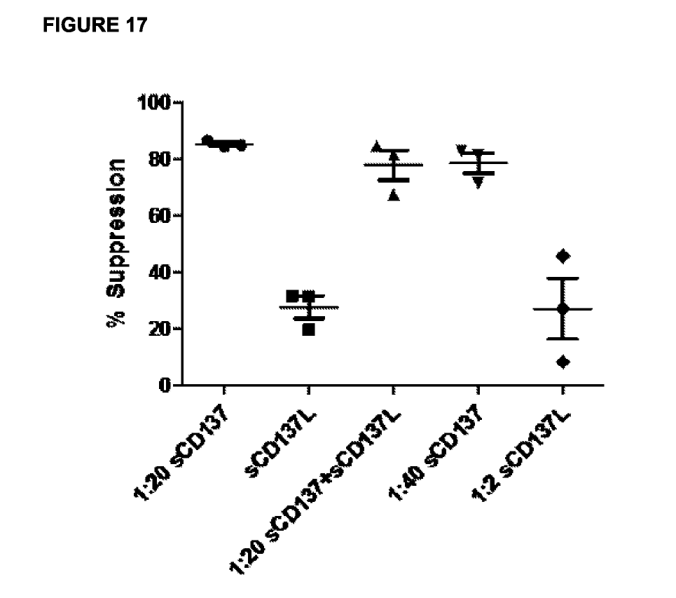

sCD137 was added together with an excess of the known CD137 ligand (CD137L) (Goodwin et al, Eur J Immunol. 1993 October; 23(10):2631-41) in soluble form to HIV-infected CD4⁺ cells and no effect of the soluble version of CD137L was observed (FIG. 17) (see also, example 5-Table 2).

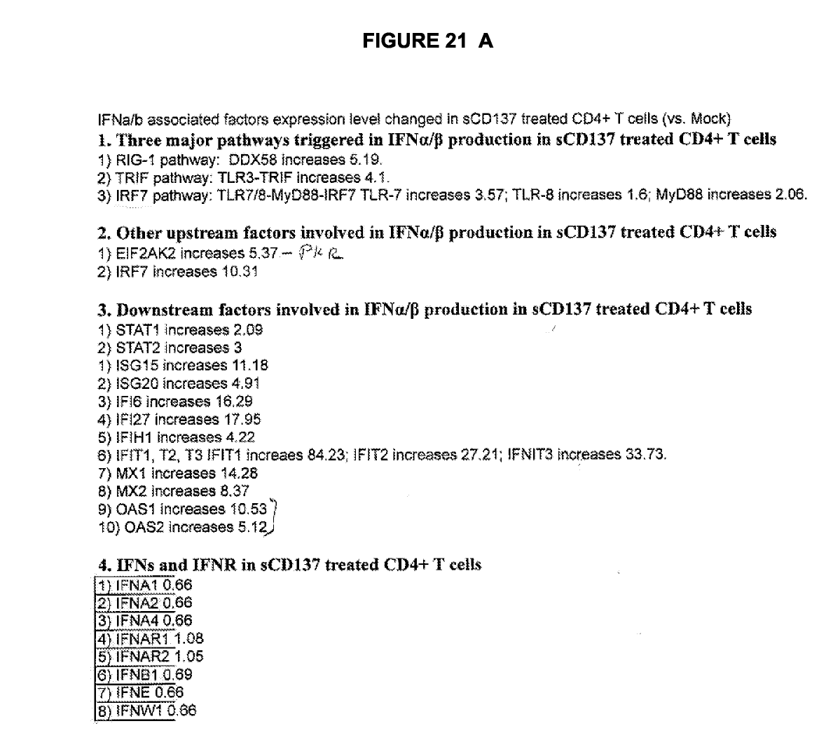

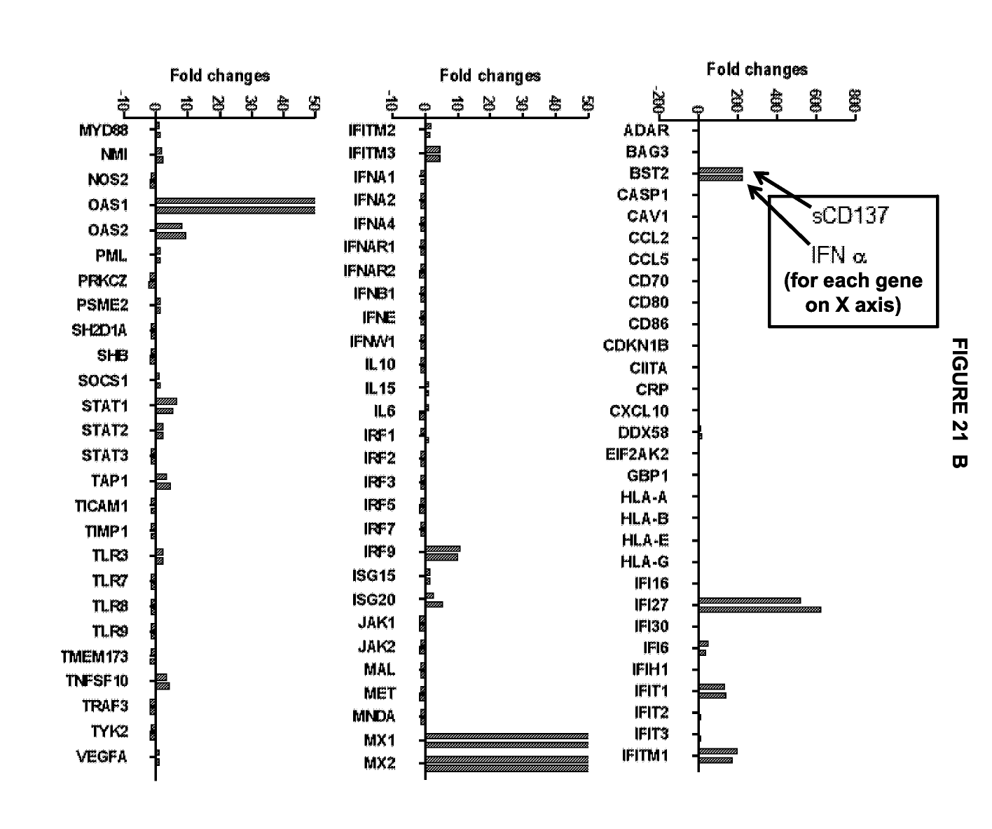

To test the effect of sCD137 on gene transcription levels, cells were contacted with sCD137 and RNA transcript levels were measured and compared to transcript levels in mock treated cells measured (FIG. 21A was CD4⁺ T cells and FIG. 21B was HeLa cells). FIGS. 21A and 21B show that the block of viral activity by sCD137 resembled that induced by interferon-α.

### Example 2

sCD137 Inhibits the Activity of a Wide Variety of Viruses in Multiple Different Cell Types

Cytomegalovirus. The addition of sCD137 (5 μg/ml) to cells infected with cytomegalovirus (CMV) reduced CMV replication in human foreskin fibroblast cells by about 75% (i.e., 75% suppression). In a 96-well plate, 0.05×10⁵ human foreskin fibroblasts (HuF cells) were cultured in 200 μL Dulbecco's Modified Eagle Medium (DMEM) supplemented with 10% fetal bovine serum (FBS) and 1× penicillin-streptomycin. Cells were cultured overnight at 36° Celsius in 5% CO₂. On the following day, all media were removed from each culture and rinsed twice with phosphate buffered saline (PBS) (Ca2+ and Mg2+ free). CMV AD 169 (2.5×10⁶ particles/ml) was diluted 1:5,000 in DMEM supplemented with 1% FBS. 200 μL of virus dilution was added to each culture. Cells were incubated with virus for 3 hours. After infection, media were removed and cells were washed twice with PBS. 100 μL of fresh HuF medium and 100 μL of “test supernatant” were added to each culture. Cultures were maintained for 6 days, removing 100 μL of medium every 2 days and replacing with 50 μl HuF medium and 50 μl “test supernatant”. On the sixth day post-infection, media were removed, and cultures were washed with PBS. 4% paraformaldehyde was added to each culture for 20 minutes and kept at room temperature. Cultures were washed once with PBS, and 40 μl 0.05% crystal violet was added. Cultures were incubated for 30 minutes at room temperature and washed twice with PBS. Plates were dried and plaques were counted using a standard inverted microscope. Cultures in which “test supernatant” contained sCD137 exhibited roughly 75% fewer plaques than cultures in which “test supernatant” did not contain sCD137.

Human herpes virus HHV6. The addition of sCD137 (protein A, 5 μg/ml) to HeLa cells infected with human herpes virus 6 (HHV-6) reduced HHV-6 replication in three-day phytohemagglutinin (PHA)-stimulated CD4+ human cells by about 50%. Three-day phytohemagglutinin (PHA)-stimulated CD4+ cells were washed once with Hanks Balanced Salt Solution (HBSS). Cells were resuspended in 1× RPMI 1640 diluted HHV6 virus at 1:4, 1:40 or 1:400, and incubated for 1 hour at 36° C., shaking every 15 minutes. After infection, cells were diluted in HBSS and counted. Cells were spun down and resuspended at 1 million/mL 1× RPMI 1640 supplemented with 10% FBS, 5 mL L-glutamine and 5 mL penicillin-streptomycin (HUT media). In a 48-well plate, 0.2×10⁶ cells were added to each well per virus dilution. Equivalent volume of “test supernatant” was added to each culture. Cultures were passed every 2-3 days removing 200 μl of medium and adding 100 μl of HUT media supplemented with 20 U interleukin-2 and 100 μl of “test supernatant”. On the 7th day post-infection, cells were counted and analyzed via immunofluorescence assay (IFA). Cultures in which “test supernatant” contained sCD137 exhibited roughly 50% lower counts (measured by IFA) than cultures in which “test supernatant” did not contain sCD137.

For the immunofluorescence assay (IFA) above, cells were resuspended at 0.5×10⁶ per ml of cold Dulbecco's Phosphate Buffered Saline (DPBS). 5,000 cells were plated per well of a 12-well slide. Plated cells were allowed to air dry completely, and fixed in cold acetone for 15 minutes at 4° C. Cells were permeabilized with DPBS containing 0.1% Triton X-100 for 3 minutes at 4° Celsius, and blocked with 7.5% BSA in PBS for 1 hour at room temperature or overnight at 4° C. Cells were incubated with primary antibody (15 μl purified ascites HHV6-P180 [4A6] from NIH into 50 mL PBS with 0.5% bovine serum albumin [BSA]) for 2 hours at room temperature or overnight at 4° Celsius. Cells were washed 3 times with DPBS at room temperature for 5 minute followed by incubation with Goat anti-Human IgG FITC-conjugated (1:100 in DPBS) for 1 hour at room temperature. The nuclei were counterstained with 50 μl of Vectashield mounting media and visualized using a fluorescence microscope.

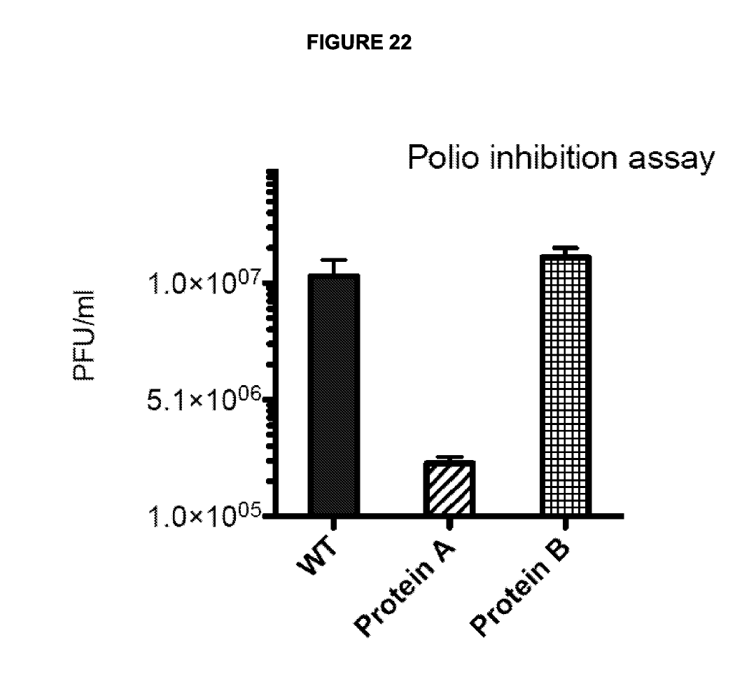

Poliovirus (a picornavirus). The addition of sCD137 (Protein A, 150 ng/ml) to HeLa cells infected with poliovirus reduced poliovirus activity compared to the addition of mock protein (protein B) (FIG. 22). Briefly, poliovirus was added to HeLa cells at low multiplicity of infection (MOI˜0.1) for 1 hour. Virus was washed out of the solution using 3 PBS washes. Virus-infected cells were incubated with Protein A (sCD137), Protein B (mock protein), or no protein (“WT”) for 8 hours. Viral titer was then measured. 3 replicates were performed for each sample. The results showed that control infected cells (either exposed to the mock protein (Protein B) or not treated (“WT”)) had a higher viral titer than cells treated with Protein A (sCD137).

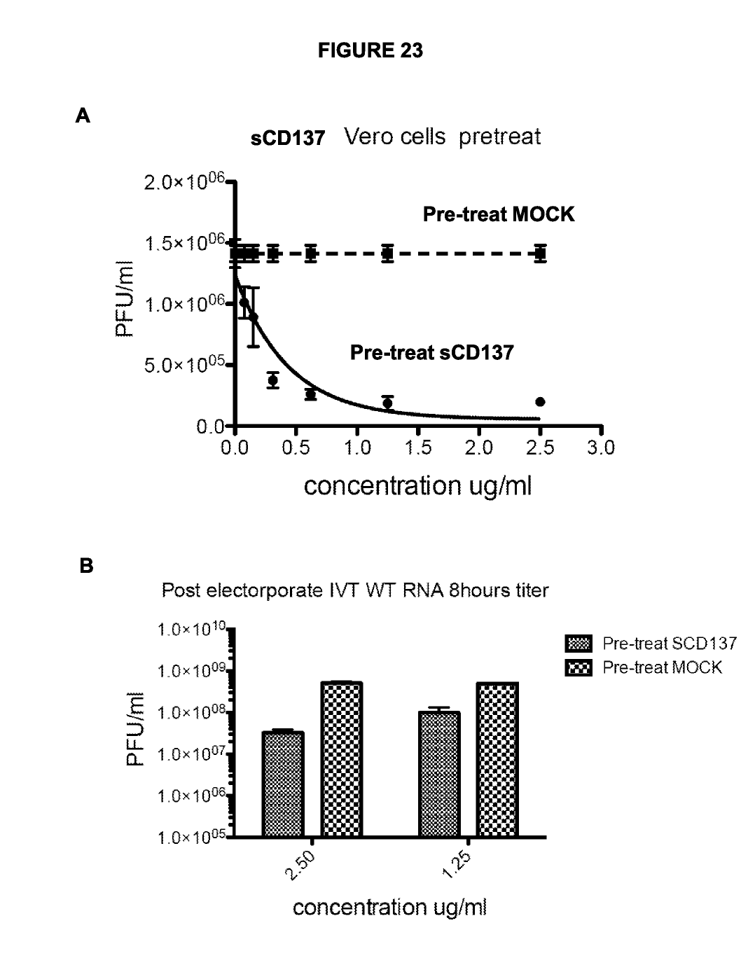

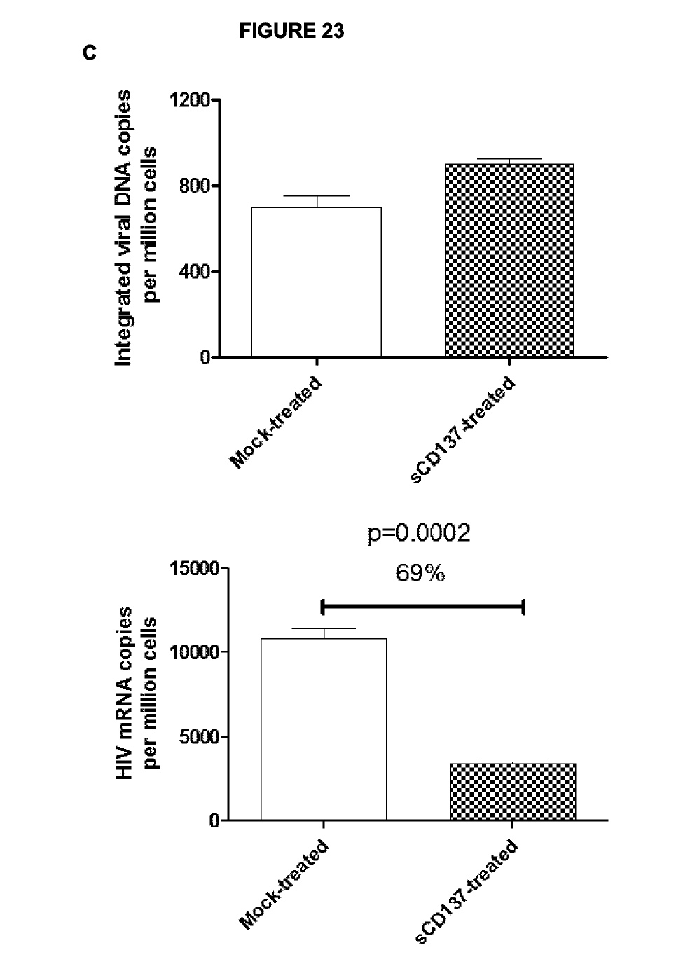

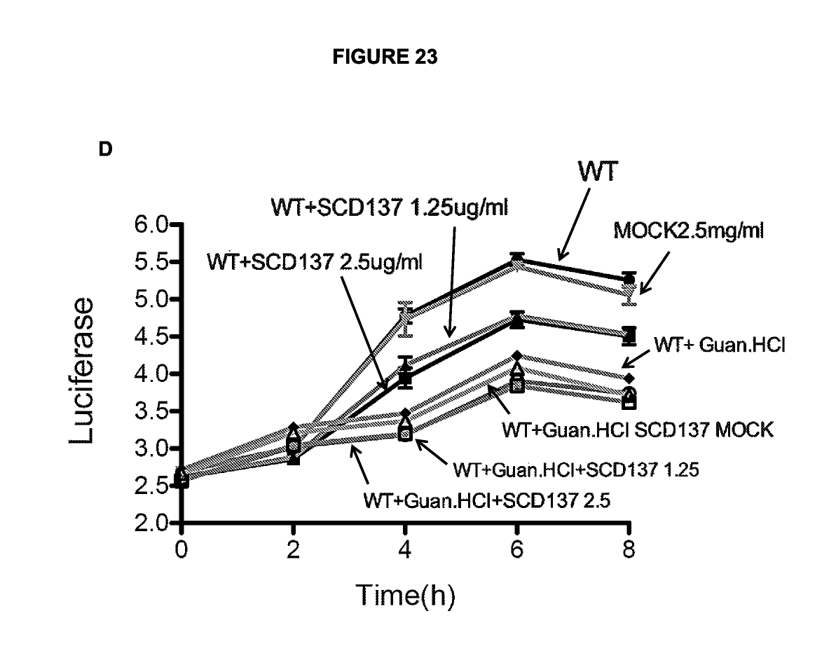

FIG. 23 depicts inhibition of polio virus activity in Vero cells (African green monkey kidney cells) pre-treated for 4 hours with sCD137. The results suggest that sCD137 mediated viral inhibition is likely to work in all primate cell types (e.g., human cells, non-human primate cells, monkey cells, etc.).

FIG. 23A: Vero cells were pretreated for 4 hours with mock (control) or with sCD137, and then challenged with polio virus.

FIG. 23B: To test whether sCD137 blocks viral entry, or instead blocks at a step downstream of viral entry, in vitro transcribed RNA of Polio Mahoney WT virus was electroporated into Vero cells that were pretreated either with mock (control) or with sCD137. Plaque assays were performed 8 hours after electroporation. The data show that pre-treatment with sCD137 still inhibits the virus (viral titer was roughly 10-fold less when cells were pre-treated with 2.5 μg/ml sCD137compared to mock-treated cells), even when the viral RNA is directly electroporated into the cells. Thus, viral entry is likely not the step at which sCD137 inhibits viruses.

FIG. 23C: shows that sCD137 treatment did not block HIV viral DNA integration into the host cell genome when viral nucleic acid was directly transfected into cells (e.g., via electroporation) (top panel), but that sCD137 still blocked viral activity, as detected by measuring viral mRNA production (bottom panel). Thus, treatment with sCD137 blocked the viral replicative cycle.

FIG. 23D: The ability of sCD137 to block Polio viral activity was tested in the presence or absence of an RNA synthesis inhibitor (Guanidinium hydrochloride, a potent inhibitor of poliovirus replication). HelaS3 cells were pre-treated with SCD137, Mock, or 2% newborn calf serum (NCS) medium. After three washes with phosphate buffered saline (PBS) cells were electroporated to introduce an in vitro transcribed RNA Polio-Luciferase replicon. The eletroporated cells were then cultured either in 2% NCS medium or in the presence of guanidinium hydrochloride (Guan.HCl). In the presence of guanidinium hydrochloride, translation can occur but RNA synthesis cannot. FIG. 23C shows that in the absence of Guan.Hcl, luciferase activity (which represents viral activity in this case, and is a measure of translation of the electroporated luciferase mRNA) of the mock group was 15 times higher than the SCD137 treated group. Thus, sCD137 inhibited viral protein production in the absence of Guan.HCl. However, in the presence of guanidinium hydrochloride (and therefore in the absence of RNA synthesis), sCD137 did not inhibit viral protein production (luciferase translation). The conclusion is that sCD137 does not block viral activity by blocking translation, and instead likely blocks transcription.

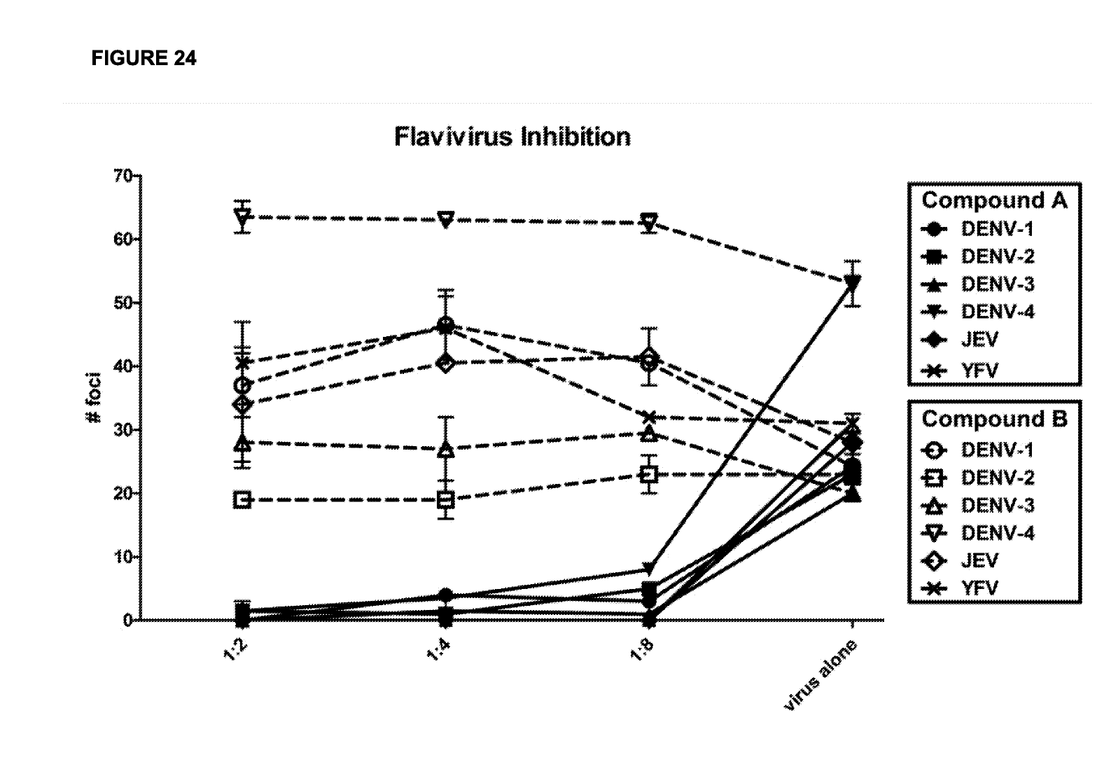

Flaviviruses. FIG. 24 depicts data showing that sCD137 (1 μg/ml) inhibited the activity of multiple different flaviviruses in Vero cells (African green monkey kidney cells). Flaviviruses tested: Dengue Virus (DENV-1, -2, -3, and -4); Japanese Encephalitis Virus (JEV); and Yellow Fever Virus (YFV). Compound A is sCD137 and compound B is control. Y-axis is the number of viral foci counted. In the presence of sCD137 (compound A), and in a concentration dependent manner, viral foci were greatly reduced.

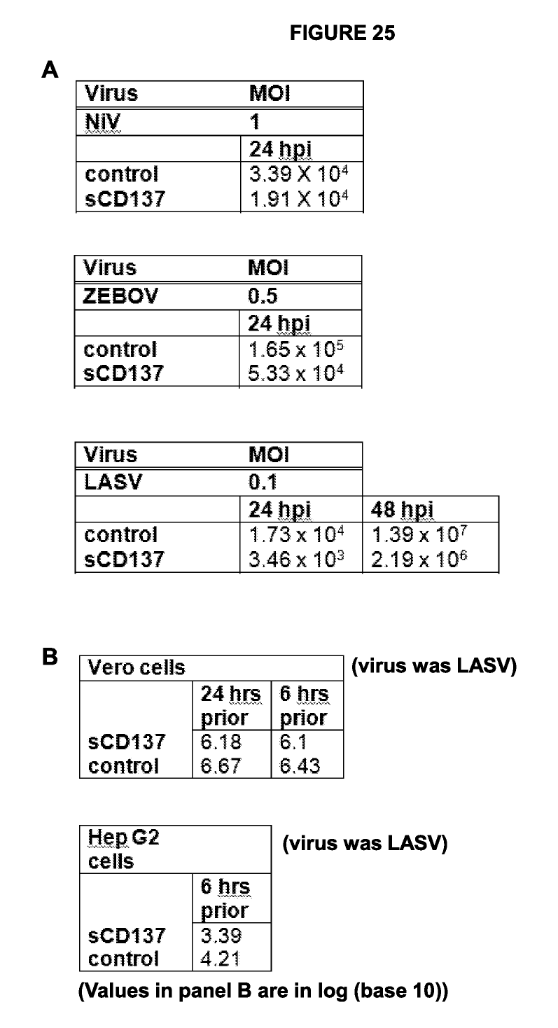

Paramyxoviruses, Filoviruses, Arenoviruses FIG. 25 depicts data showing that sCD137 (2.5 μg/ml) inhibits Nipah virus (NiV) virus activity, Lassa virus (LASV) virus activity, and ebola virus (ZEBOV) virus activity in Human Umbilical Vein Endothelial Cells (HUVECs); and LASV activity in Vero cells and human Hep G2 cells. (A) HUVECs were pretreated for 6 hours with sCD137 or control (mock fluid) prior to being innoculated with NiV, LASV, or ZEBOV. Results are in units of PFU/ml (plaque forming units per ml). MOI (Multiplicity of Infection). Plaques were counted 24 or 48 hpi (hours post transfection). (B) Vero Cells and Hep G2 cells were pretreated for 6 hours or 24 hours with sCD137 or control prior to being contacted with LASV virus. Results are in log 10 (counts were of plaques and cells were stained with neutral red). MOI (Multiplicity of Infection) was 0.1 for both cell types. Plaques were counted 48 hpi (hours post transfection). Values are in log (base 10).

Rotavirus. The addition of sCD137 (100 ng/ml) to HT-29 cells (human adherent epithelial colorectal adenocarcinoma cells) suppressed viral activity of the rotavirus by 50%.

Rhinovirus. sCD137 inhibited rhinovirus replication in HeLa cells as assayed using a plaque assay.

See Table 1 (above), which summarizes results showing that sCD137 inhibits the activity of a wide variety of viruses in multiple different cell types.

### Example 3

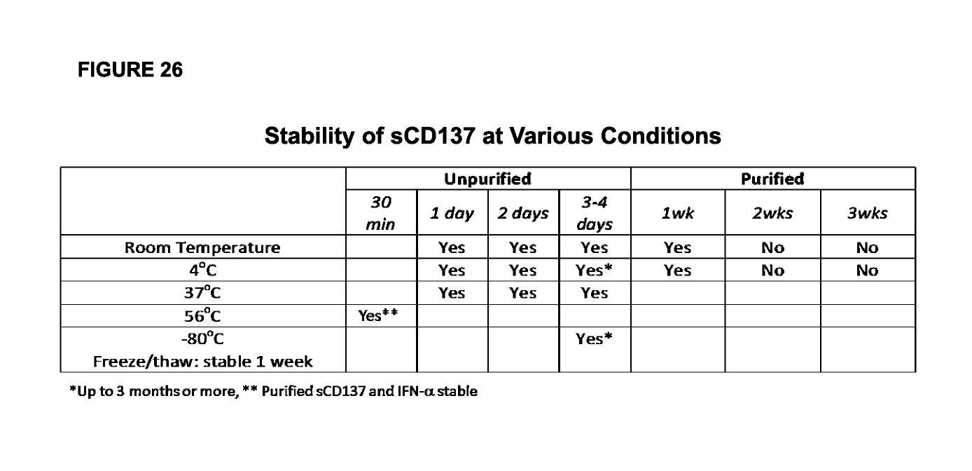

sCD137 was stable at different temperatures (FIG. 26).

### Example 4

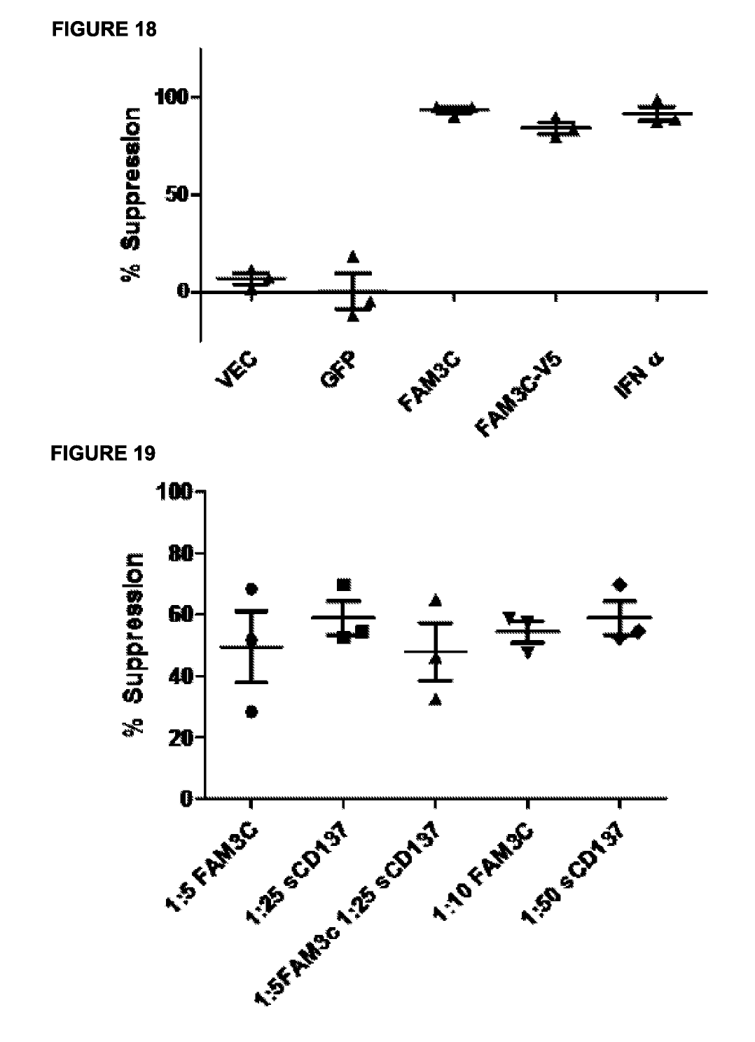

FAM3C and FAM3C fused to a V5 tag (FAM3C-v5) were also shown to suppress HIV infected CD4⁺ cells (FIG. 18). Interferon-α was used as a positive control. Mixing sCD137 and FAM3C did not change the antiviral activity of either one of the proteins (FIG. 19).

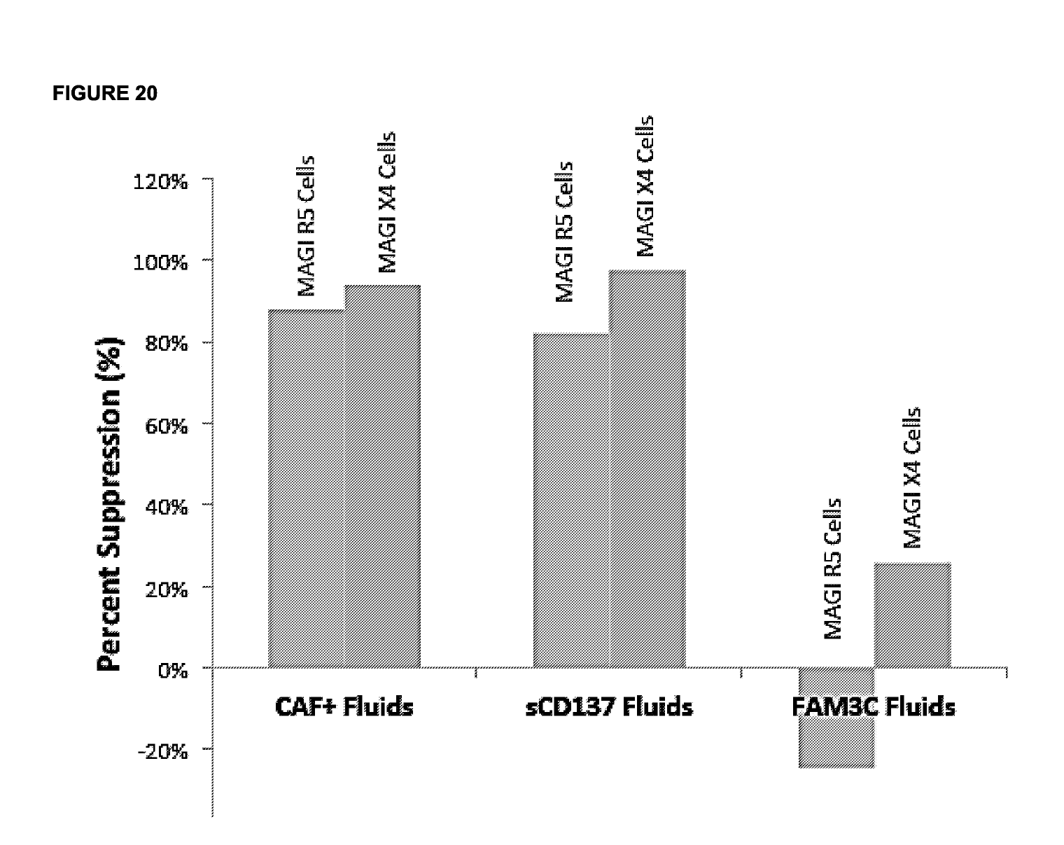

When human U373 glioblastoma cells (MAGI) (susceptible to either R5-tropic or X4-tropic HIV) were infected with HIV (R5-tropic or X4-tropic) and then supernatant was added, virus replication was inhibited by the sCD137-containing supernatant, but not by the FAM3C-containing supernatant (FIG. 20).

Inhibition using FAM3C was most prominent when primary CD4⁺ cells were pre-treated with FAM3C for two hours prior to infecting the CD4⁺ cells with HIV.

### Example 5

Because the interferon pathway appeared to be induced after pre-treatment of cells with sCD137, a block of the antiviral effect was attempted with shRNA to IRF-3. None was observed.

An antibody (anti-CD137L, “CD137L-antibody”) to the known ligand for CD137 (CD137L), was used to determine its effect on the induction of the antiviral state by sCD137 and type-1 interferon (IFN). The antibody (Ab) blocked the effect of sCD137 but not IFN (Table 2). This observation indicated that, whereas the soluble version of CD137L did not block the activity of sCD137 (see example 1, FIG. 17), contacting CD137L on the CD4+ cell surface with an anti-CD137L antibody did prevent the sCD137 induced antiviral state in these cells.

In brief, sCD137 protein was purified. On day 1, NL20123 CD4 cells were treated with anti-CD137L antibody (“CD137L-antibody”) (20 ug/ml) (or controls) for 1 hour. Treated cells were then infected by SF2 (1:10) (on Day 1). Infected cells were then incubated with the anti-CD137L antibody (“CD137L-antibody”) (or controls) plus: (i) 300 ng/ml sCD137, (ii) 500 U/ml IFNa or (iii) two different types of controls (Hut or Mock) for 4 days. (On Day 2, culture was changed). On day 4, HIV p24 ELISA was performed. The results are presented in Table 2.

TABLE 2
Results from experiment to test whether an anti-CD137L antibody
can block the anti-viral effect of sCD137 and/or interferon (IFNa).

| | | Mean | | | % |
|---|---|---|---|---|---|
| | Result | Result | Std. Dev. | CV % | Suppression |
| No antibody + | 909.466 | 874.804 | 66.795 | 7.6 | |
| Mock | 917.142 | | | | |
| | 797.803 | | | | |
| No antibody + | 291.131 | 376.829 | 76.363 | 20.3 | 56.92 |
| sCD137 | 401.699 | | | | |
| | 437.656 | | | | |
| No antibody + | 961.278 | 808.533 | 164.149 | 20.3 | |
| Hut | 634.966 | | | | |
| | 829.354 | | | | |
| No antibody + | 144.93 | 198.603 | 46.687 | 23.5 | 75.44 |
| IFNa | 221.064 | | | | |
| | 229.814 | | | | |
| Control + | 792.6 | 826.122 | 97.627 | 11.8 | |
| Mock | 936.093 | | | | |
| | 749.672 | | | | |
| Control + | 364.771 | 368.114 | 97.430 | 26.5 | 55.44 |
| sCD137 | 272.399 | | | | |
| | 467.173 | | | | |
| Control + | 838.391 | 911.873 | 68.449 | 7.5 | |
| Hut | 923.401 | | | | |
| | 973.826 | | | | |
| Control + | 167.433 | 194.269 | 23.317 | 12 | 78.70 |
| IFNa | 209.578 | | | | |
| | 205.795 | | | | |
| CD137L- | 845.578 | 851.542 | 139.269 | 16.4 | |
| antibody + | 715.35 | | | | |
| Mock | 993.697 | | | | |
| CD137L- | 689.619 | 735.836 | 115.234 | 15.7 | 13.59 |
| antibody + | 867.004 | | | | |
| sCD137 | 650.884 | | | | |
| CD137L- | 720.246 | 950.853 | 247.719 | 26.1 | |
| antibody + | 1212.717 | | | | |
| Hut | 919.595 | | | | |
| CD137L- | 188.133 | 213.969 | 22.46 | 10.5 | 77.50 |
| antibody + | 228.836 | | | | |
| IFNa | 224.939 | | | | |

Antibodies to interferon (at 1 μg/ml) were mixed with purified sCD137 (300 ng/ml) and 500 U of IFN along with relative controls for 1 hour at 37° C. The mixture was then added to CD4+ T cells acutely infected with HIV-1. Cultures were changed at day 2 and fresh antibody to interferon with the sCD137 or with IFN was added. The results (from HIV p24 ELISA) indicated that there is no effect of the interferon antibody on sCD137 but it blocked the antiviral effect of IFN (Table 3).

TABLE 3
Results from experiment to test whether an anti-IFNa antibody
can block the anti-viral effect of sCD137 and/or interferon (IFNa).

| | | Mean | | | % |
|---|---|---|---|---|---|
| | Result | Result | Std. Dev. | CV % | Suppression |
| Mock for sCD137 | 365.0776 | 415.81 | 98.76 | 23.75 | |
| | 352.7198 | | | | |
| | 529.6196 | | | | |
| sCD137 | 193.8691 | 185.82 | 11.21 | 6.03 | 55.31 |
| | 190.5853 | | | | |
| | 173.018 | | | | |
| Mock for IFNa | 473.7017 | 394.53 | 83.16 | 21.08 | |
| | 402.0126 | | | | |
| | 307.8857 | | | | |
| IFNa 500 U/ml | 30.8476 | 39.75 | 14.55 | 36.59 | 89.92 |
| | 31.87 | | | | |
| | 56.5373 | | | | |
| Mock for | 380.9364 | 421.50 | 35.41 | 8.40 | |
| sCD137 + Control | 446.2141 | | | | |
| antibody | 437.3444 | | | | |
| sCD137 + | 164.8524 | 209.64 | 42.72 | 20.38 | 50.26 |
| Control antibody | 249.9423 | | | | |
| | 214.1323 | | | | |
| Mock for IFNa + | 342.6477 | 374.01 | 78.26 | 20.93 | |
| Control antibody | 463.0839 | | | | |
| | 316.2856 | | | | |
| IFNa 500 U/ml + | 18.4891 | 28.43 | 9.04 | 31.80 | 92.40 |
| Control antibody | 30.6276 | | | | |
| | 36.1637 | | | | |
| Mock for | 515.6964 | 474.25 | 44.62 | 9.41 | |
| sCD137 + IFNa | 480.0365 | | | | |
| antibody | 427.0174 | | | | |
| sCD137 + | 211.4406 | 231.89 | 20.09 | 8.66 | 51.10 |
| IFNa antibody | 232.6478 | | | | |
| | 251.5933 | | | | |
| Mock for IFNa + | 456.5284 | 452.92 | 69.64 | 15.38 | |
| IFNa antibody | 381.5475 | | | | |
| | 520.6884 | | | | |
| IFNa 500 U/ml + | 330.5039 | 344.17 | 22.33 | 6.49 | 24.01 |
| IFNa antibody | 332.0691 | | | | |
| | 369.9348 | | | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inhibiting the activity of a virus in an individual, the method comprising:
administering to the individual an effective amount of an anti-viral composition comprising at least one anti-viral agent selected from: an sCD137 polypeptide, a nucleic acid comprising a nucleotide sequence that encodes an sCD137 polypeptide, a FAM3C polypeptide, and a nucleic acid comprising a nucleotide sequence that encodes a FAM3C polypeptide,
wherein the individual is infected with the virus, has an increased risk of being infected with the virus, or is suspected of being infected with the virus.

2. The method of claim 1, wherein the virus is a virus selected from the group consisting of: HIV (Human Immunodeficiency Virus), poliovirus, RSV (human respiratory syncytial virus), EBV (Epstein-Barr virus), HSV (herpes simplex virus), CMV (cytomegalovirus), Dengue Virus, JEV (Japanese Encephalitis Virus), and influenza virus.

3. The method of claim 1, wherein the virus is a virus selected from the group consisting of: a picornavirus, a herpesvirus, a myxovirus, a paramyxovirus, an orthomyxovirus, a rotavirus, a rhinovirus, and a flavivirus.

4. The method of claim 1, wherein the virus is a virus selected from the group consisting of: HIV (Human Immunodeficiency Virus)-1, HIV-2, DENV (dengue virus)-2, DENV-2, DENV-3, DENV-4, YFV (yellow fever virus), JEV (Japanese encephalitis virus), poliovirus, rhinovirus, coxsackievirus, NiV (Nipah virus), ZEBOV (Zaire ebola virus), CMV (cytomegalovirus), HHV (Human herpes virus)-6, and LASV (Lassa virus).

5. The method of any of claims 1-4, wherein the sCD137 polypeptide comprises an amino acid sequence having 60% or more amino acid sequence identity to amino acids 1-138 of an amino acid sequence set forth in any of SEQ ID NOs: 1, 20, and 22-27.

6. The method of any of claims 1-5, wherein the FAM3C polypeptide comprises an amino acid sequence having 75% or more amino acid sequence identity to an amino acid sequence set forth in any of SEQ ID NOs: 3 and 28.

7. The method of any of claims 1-6, wherein the contacting occurs in vivo.

8. The method of any of claims 1-6, wherein the contacting occurs ex vivo.

9. The method of any of claims 1-8, wherein the individual is a human.

10. The method of any of claims 1-9, wherein the nucleotide sequence that encodes an sCD137 polypeptide is operably linked to a transcriptional control element that provides for expression in a CD8⁺ T lymphocyte.

11. The method of any of claims 1-10, further comprising administering to the individual at least one additional therapeutic agent.

12. The method of any of claims 1-11, further comprising a step of measuring the viral load in a biological sample obtained from the individual.

13. The method of claim 12, wherein the step of measuring the viral load comprises:
measuring the viral load in a biological sample obtained from the individual prior to the step of administering; and
measuring the viral load in a biological sample obtained from the individual after the step of administering.

14. The method of any of claims 1-13, wherein the anti-viral agent is formulated in a form suitable for intravaginal or rectal administration.

15. A method of inhibiting viral activity in a target cell that is infected with a virus, has an increased risk of acquiring a virus, or is suspected of having a virus; the method comprising at least one of:
(i) contacting the target cell with an effective amount of an anti-viral composition comprising at least one anti-viral agent selected from: an sCD137 polypeptide, and a FAM3C polypeptide; and
(ii) introducing into the cell an anti-viral composition comprising at least one anti-viral agent selected from: a nucleic acid comprising a nucleotide sequence that encodes an sCD137 polypeptide, and a nucleic acid comprising a nucleotide sequence that encodes a FAM3C polypeptide.

16. The method of claim 15, wherein the virus is a virus selected from the group consisting of: HIV (Human Immunodeficiency Virus), poliovirus, RSV (human respiratory syncytial virus), EBV (Epstein-Barr virus), HSV (herpes simplex virus), CMV (cytomegalovirus), and influenza virus.

17. The method of claim 15, wherein the virus is a virus selected from the group consisting of: a picornavirus, a herpesvirus, a myxovirus, a paramyxovirus, an orthomyxovirus, a rotavirus, a rhinovirus, and a flavivirus.

18. The method of claim 15, wherein the virus is a virus selected from the group consisting of: HIV (Human Immunodeficiency Virus)-1, HIV-2, DENV (dengue virus)-2, DENV-2, DENV-3, DENV-4, YFV (yellow fever virus), JEV (Japanese encephalitis virus), poliovirus, rhinovirus, coxsackievirus, NiV (Nipah virus), ZEBOV (Zaire ebola virus), CMV (cytomegalovirus), HHV(Human herpes virus)-6, and LASV (Lassa virus).

19. The method of any of claims 15-18, wherein the target cell is in vitro.

20. The method of any of claims 15-18, wherein the target cell is ex vivo.

21. The method of any of claims 15-18, wherein the target cell is in vivo.

22. The method of any of claims 15-21, wherein the target cell is a human cell.

23. The method of any of claims 15-22, wherein the target cell is contacted with the anti-viral composition for a period of time of up to 72 hours.

24. The method of any of claims 15-23, wherein the target cell is contacted with the anti-viral composition up to 48 hours prior to suspected potential contact with the virus, up to 4 hours after being contacted with the virus, or up to about 4 hours after suspected contact with the virus.

25. The method of any of claims 15-24, wherein the sCD137 polypeptide comprises an amino acid sequence having 60% or more amino acid sequence identity to amino acids 1-138 of an amino acid sequence set forth in any of SEQ ID NOs: 1, 20, and 22-27.

26. The method of any of claims 15-25, wherein the FAM3C polypeptide comprises an amino acid sequence having 75% or more amino acid sequence identity to an amino acid sequence set forth in any of SEQ ID NOs: 3 and 28.


SEQUENCE LISTING (28 sequences)

SEQ ID NO: 1 - protein, 255 aa, Homo Sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 2 - DNA, 765 bp, Homo Sapiens
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 3 - protein, 227 aa, Homo Sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 4 - DNA, 681 bp, Homo Sapiens
atgagggtagcaggtgctgcaaagttggtggtagctgtggcagtgtttttactgacattttatgttatttctcaagtatttgaaataaaaatggatgcaagtttaggaaatctatttgcaagatcagcattggacacagctgcacgttctacaaagcctcccagatataagtgtgggatctcaaaagcttgccctgagaagcattttgcttttaaaatggcaagtggagcagccaacgtggtgggacccaaaatctgcctggaagataatgttttaatgagtggtgttaagaataatgttggaagagggatcaatgttgccttggcaaatggaaaaacaggagaagtattagacactaaatattttgacatgtggggaggagatgtggcaccatttattgagtttctgaaggccatacaagatggaacaatagttttaatgggaacatacgatgatggagcaaccaaactcaatgatgaggcacggcggctcattgctgatttggggagcacatctattactaatcttggttttagagacaactgggtcttctgtggtgggaagggcattaagacaaaaagcccttttgaacagcacataaagaacaataaggatacaaacaaatatgaaggatggcctgaagttgtagaaatggaaggatgcatcccccagaagcaagac

SEQ ID NO: 5 - protein, 16 aa, Artificial sequence
  Synthetic amino acid sequence
RQIKIWFQNRRMKWKK

SEQ ID NO: 6 - protein, 11 aa, Artificial sequence
  Synthetic amino acid sequence
YGRKKRRQRRR

SEQ ID NO: 7 - protein, 12 aa, Artificial sequence
  Synthetic amino acid sequence
RRQRRTSKLMKR

SEQ ID NO: 8 - protein, 27 aa, Artificial sequence
  Synthetic amino acid sequence
GWTLNSAGYLLGKINLKALAALAKKIL

SEQ ID NO: 9 - protein, 33 aa, Artificial sequence
  Synthetic amino acid sequence
KALAWEAKLAKALAKALAKHLAKALAKALKCEA

SEQ ID NO: 10 - protein, 16 aa, Artificial sequence
  Synthetic amino acid sequence
RQIKIWFQNRRMKWKK

SEQ ID NO: 11 - protein, 9 aa, Artificial sequence
  Synthetic amino acid sequence
RKKRRQRRR

SEQ ID NO: 12 - protein, 8 aa, Artificial sequence
  Synthetic amino acid sequence
RKKRRQRR

SEQ ID NO: 13 - protein, 11 aa, Artificial sequence
  Synthetic amino acid sequence
YARAAARQARA

SEQ ID NO: 14 - protein, 11 aa, Artificial sequence
  Synthetic amino acid sequence
THRLPRRRRRR

SEQ ID NO: 15 - protein, 11 aa, Artificial sequence
  Synthetic amino acid sequence
GGRRARRRRRR

SEQ ID NO: 16 - protein, 36 aa, Artificial sequence
  Synthetic amino acid sequence
YTSLIHSLIEESQNQQEKNEQELLELDKWASLWNWF

SEQ ID NO: 17 - protein, 9 aa, Artificial sequence
  Synthetic amino acid sequence
YPYDVPDYA

SEQ ID NO: 18 - protein, 8 aa, Artificial sequence
  Synthetic amino acid sequence
DYKDDDDK

SEQ ID NO: 19 - protein, 10 aa, Artificial sequence
  Synthetic amino acid sequence
EQKLISEEDL

SEQ ID NO: 20 - protein, 190 aa, Artificial sequence
  Synthetic amino acid sequence
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISF

SEQ ID NO: 21 - DNA, 573 bp, Artificial sequence
  Synthetic nucleic acid sequence
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctag

SEQ ID NO: 22 - protein, 211 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPAFKKTTGAAQEEDACSCRCPQEEEGGGGGYEL

SEQ ID NO: 23 - protein, 138 aa, Artificial Sequence
  Synthetic amino acid sequence
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTN

SEQ ID NO: 24 - protein, 205 aa, Artificial Sequence
  Synthetic amino acid sequence
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPAFKKTTGAAQEEDACSCRCPQEEEGG

SEQ ID NO: 25 - protein, 252 aa, Artificial Sequence
  Synthetic amino acid sequence
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGG

SEQ ID NO: 26 - protein, 165 aa, Artificial Sequence
  Synthetic amino acid sequence
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSF

SEQ ID NO: 27 - protein, 138 aa, Artificial Sequence
  Synthetic amino acid sequence
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTN

SEQ ID NO: 28 - protein, 227 aa, Mus musculus
MRVAGAAKLVVAVAVFLLTFYVISQVFEIKMDASLGNLFARSALDSAIRSTKPPRYKCGISKACPEKHFAFKMASGAANVVGPKICLEDNVLMSGVKNNVGRGINIALVNGKTGEVIDTKFFDMWGGDVAPFIEFLKTIQDGTVVLMATYDDGATKLTDEARRLIAELGSTSITSLGFRDNWVFCGGKGIKTKSPFEQHIKNNKETNKYEGWPEVVEMEGCIPQKQD